SEQUENCE LISTING (47 sequences)

SEQ ID NO: 1 - protein, 97 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 2 - protein, 104 aa, Staphylococcus aureus
MGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 3 - protein, 302 aa, Staphylococcus aureus
MKKLLLPLIIMLLVLAACGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 4 - protein, 256 aa, Staphylococcus aureus
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 5 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 6 - protein, 285 aa, Staphylococcus aureus
CGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 7 - protein, 288 aa, Staphylococcus aureus
MASCGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 8 - protein, 234 aa, Staphylococcus aureus
MGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 9 - protein, 256 aa, Staphylococcus aureus
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 10 - protein, 256 aa, Staphylococcus aureus
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKVKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 11 - protein, 256 aa, Staphylococcus aureus
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNVKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 12 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 13 - protein, 293 aa, Staphylococcus aureus
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 14 - protein, 4 aa, Staphylococcus aureus
PSGS

SEQ ID NO: 15 - protein, 258 aa, Staphylococcus aureus
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTPSGSVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 16 - protein, 258 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTPSGSVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 17 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDLYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 18 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPLFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 19 - protein, 293 aa, Staphylococcus aureus
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDLYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 20 - protein, 293 aa, Staphylococcus aureus
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPLFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 21 - protein, 50 aa, Staphylococcus aureus
ADSDINIKTGTTDIGSNTTVKTGDLVTYDKENGMHKKVFYSFIDDKNHNK

SEQ ID NO: 22 - protein, 63 aa, Staphylococcus aureus
ADSDINIKTGTTDIGSNTTVKTGDLVTYDKENGMHKKVFYSFIDDKNHNKKLLVIRTKGTIAG

SEQ ID NO: 23 - protein, 53 aa, Staphylococcus aureus
ADSDINIKTGTTDIGSNTTVKTGDLVTYDKENGMHKKVFYSFIDDKNHNKKLL

SEQ ID NO: 24 - protein, 50 aa, Staphylococcus aureus
ADSDINIKTGTTDIGSNTTVKTGDLVTYDKENGMLKKVFYSFIDDKNHNK

SEQ ID NO: 25 - protein, 63 aa, Staphylococcus aureus
ADSDINIKTGTTDIGSNTTVKTGDLVTYDKENGMLKKVFYSFIDDKNHNKKLLVIRTKGTIAG

SEQ ID NO: 26 - protein, 53 aa, Staphylococcus aureus
ADSDINIKTGTTDIGSNTTVKTGDLVTYDKENGMLKKVFYSFIDDKNHNKKLL

SEQ ID NO: 27 - protein, 296 aa, Staphylococcus aureus
MASADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 28 - protein, 207 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSMGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 29 - protein, 207 aa, Staphylococcus aureus
MGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNPASGGGSMAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 30 - protein, 6 aa, Artificial Sequence
  Linker
ASGGGS

SEQ ID NO: 31 - protein, 205 aa, Staphylococcus aureus
AMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 32 - protein, 206 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 33 - protein, 233 aa, Staphylococcus aureus
GCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 34 - protein, 927 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSDSDSDSDSDSDSDSDSDSASDSDSGSDSDSSSDSDSESDSNSDSESVSNNNVVPPNSPKNGTNASNKNEAKDSKEPLPDTGSEDEANTSLIWGLLASIGSLLLFRRKKENKDKK

SEQ ID NO: 35 - protein, 350 aa, Staphylococcus aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 36 - protein, 145 aa, Staphylococcus aureus
DSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLA

SEQ ID NO: 37 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 38 - protein, 227 aa, Staphylococcus aureus
MKNILKVFNTTILALIIIIATFSNSANAADSGTLNYEVYKYNTNDTSIANDYFNKPAKYIKKNGKLYVQITVNHSHWITGMSIEGHKENIISKNTAKDERTSEFEVSKLNGKIDGKIDVYIDEKVNGKPFKYDHHYNITYKFNGPTDVAGANAPGKDDKNSASGSDKGSDGTTTGQSESNSSNKDKVENPQTNAGTPAYIYAIPVASLALLIAITLFVRKKSKGNVE

SEQ ID NO: 39 - protein, 895 aa, Staphylococcus aureus
MNKHHPKLRSFYSIRKSTLGVASVIVSTLFLITSQHQAQAAENTNTSDKISENQNNNATTTQPPKDTNQTQPATQPANTAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDKDADNSVGMSSNVDTDKDSNKNKDKVIQLNHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 40 - protein, 520 aa, Staphylococcus aureus
SENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 41 - protein, 284 aa, Staphylococcus aureus
GNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 42 - protein, 103 aa, Staphylococcus aureus
  misc_feature 30: ′Xaa′ is absent or a residue without a free thiol group
GGYKGIKADGGKVDQAKQLAAKTAKDIEAXQKQTQQLAEYIEGSDWEGQFANKVKDVLLIMAKFQEELVQPMADHQKAIDNLSQNLAKYDTLSIKQGLDRVNP

SEQ ID NO: 43 - protein, 231 aa, Staphylococcus aureus
GIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 44 - protein, 287 aa, Staphylococcus aureus
MASGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 45 - protein, 206 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEAAQKQTQQLAEYIEGSDWEGQFANKVKDVLLIMAKFQEELVQPMADHQKAIDNLSQNLAKYDTLSIKQGLDRVNP

SEQ ID NO: 46 - protein, 234 aa, Staphylococcus aureus
MGSGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 47 - protein, 205 aa, Staphylococcus aureus
  misc_feature 132: ′Xaa′ is absent or a residue without a free thiol group
AMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEAXQKQTQQLAEYIEGSDWEGQFANKVKDVLLIMAKFQEELVQPMADHQKAIDNLSQNLAKYDTLSIKQGLDRVNP

(19) United States
(12) Patent Application Publication
(10) Pub. No.: US 2014/0363461 A1
(43) Pub. Date: Dec. 11, 2014

(54) ADJUVANTED FORMULATIONS OF STAPHYLOCOCCUS AUREUS ANTIGENS

(71) Applicants: Fabio Bagnoli, Siena (IT); Barbara Baudner, Siena (IT); Simone Bufali, Siena (IT)

(72) Inventors: Fabio Bagnoli, Siena (IT); Barbara Baudner, Siena (IT); Simone Bufali, Siena (IT)

(21) Appl. No.: 14/240,616

(22) PCT Filed: Aug. 31, 2012
(86) PCT No.: PCT/EP2012/067032

§ 371 Date: Aug. 22, 2014

Related U.S. Application Data
(60) Provisional application No. 61/607,999, filed on Mar. 7, 2012.
(60) Provisional application No. 61/530,162, filed on Sep. 1, 2011.

(51) Int. Cl. A61K 39/085; A61K 39/39

(52) U.S. Cl. CPC A61K 39/085; A61K 39/39; A61K 2039/55511
USPC 424/190.1

(57) ABSTRACT

The efficacy of S. aureus vaccines can be enhanced by adjuvanting S. aureus antigens with a mixture of a TLR agonist (preferably a TLR7 agonist) and an insoluble metal salt (preferably an aluminium salt). The TLR agonist is typically adsorbed to the metal salt. A S. aureus antigen can also be adsorbed to the metal salt.

This application claims the benefit of U.S. provisional applications 61/530,162 (filed Sep. 1, 2011) and 61/607,999 (filed Mar. 7, 2012), the complete contents of both of which are hereby incorporated herein by reference for all purposes.

### TECHNICAL FIELD

The invention is in the field of adjuvanting antigens from Staphylococcus aureus to increase their immunogenicity.

### BACKGROUND ART

Reference 1 discloses various immunogens and combinations for preparing efficacious vaccines against S. aureus. Table 2 in reference 1 shows that these immunogens and combinations were adjuvanted with aluminium hydroxide or with the MF59 oil-in-water emulsion. The experimental section discloses details of adsorption studies using aluminium hydroxide.

It is an object of the invention to provide further adjuvanted immunogenic compositions for protecting against S. aureus, and in particular to provide compositions which are superior to those adjuvanted with aluminium hydroxide. Improved adjuvant effects are particularly useful for achieving rapid and robust immune responses in individuals at higher risk of S. aureus infection e.g. those preparing for surgical procedures, the immunocompromised, or the elderly.

### DISCLOSURE OF THE INVENTION

The inventors have found that the efficacy of S. aureus vaccines can be enhanced by adjuvanting S. aureus antigens with a mixture of a TLR agonist (preferably a TLR7 agonist, such as compound ‘K2’ identified below) and an insoluble metal salt (preferably an aluminium salt, such as an aluminium hydroxide). The TLR agonist is typically adsorbed to the metal salt, as disclosed in reference 2. A S. aureus antigen can also be adsorbed to the metal salt.

In a first aspect, the invention provides an immunogenic composition comprising (i) a TLR agonist (ii) an insoluble metal salt and (iii) two or more S. aureus antigens.

In a second aspect, the invention provides an immunogenic composition comprising (i) a TLR7 agonist (ii) an insoluble metal salt and (iii) at least one S. aureus antigen.

In a third aspect, the invention provides an immunogenic composition comprising (i) a TLR agonist (ii) an insoluble aluminium salt and (iii) at least one S. aureus antigen.

In a fourth aspect, the invention provides an immunogenic composition comprising (i) a TLR agonist (ii) an insoluble metal salt and (iii) a fusion protein comprising an EsxA antigen and an EsxB antigen.

In a fifth aspect, the invention provides an immunogenic composition comprising (i) a TLR agonist (ii) an insoluble metal salt and (iii) a mutant S. aureus hemolysin.

In a sixth aspect, the invention provides an immunogenic composition comprising (i) a TLR agonist (ii) an insoluble metal salt (iii) a buffer and (iv) at least one S. aureus antigen.

In a seventh aspect, the invention provides an immunogenic composition comprising (i) a TLR agonist (ii) an insoluble metal salt and (iii) at least one S. aureus antigen, wherein the composition has a pH between 6 and 8.

In an eighth aspect, the invention provides a process for preparing an immunogenic composition, wherein the process comprises mixing a TLR agonist, an insoluble metal salt, and S. aureus antigen(s), thereby providing the immunogenic composition as defined above.

In a ninth aspect, the invention provides a process for preparing an immunogenic composition, comprising one of: (i) combining a S. aureus antigen with a mixture comprising a TLR agonist and an insoluble metal salt; (ii) combining an insoluble metal salt with a mixture comprising a TLR agonist and a S. aureus antigen; or (iii) combining a TLR agonist with a mixture comprising an insoluble metal salt and a S. aureus antigen.

In a tenth aspect, the invention provides a composition comprising: (a) an adjuvant complex comprising a first TLR agonist adsorbed to an insoluble metal salt; (b) an adjuvant complex comprising a second TLR agonist adsorbed to an insoluble metal salt; and (c) at least one S. aureus antigen. The antigen(s) may be adsorbed to the metal salt(s).

In an eleventh aspect, the invention provides a process for preparing an immunogenic composition comprising steps of (i) preparing an aqueous mixture of a TLR agonist and a soluble aluminium salt, and then adding a non-aluminium salt to the aqueous mixture) in order to form a precipitated aluminium salt to which the TLR agonist is adsorbed; and (ii) mixing a S. aureus antigen with the precipitated salt and its adsorbed agonist. The TLR agonist is preferably a TLR agonist as variously described herein.

In a twelfth aspect, the invention provides a process for preparing an immunogenic composition, comprising a step of mixing (i) an aqueous mixture of a TLR agonist and a soluble aluminium salt with (ii) a buffered aqueous mixture of a S. aureus immunogen, wherein the mixing step causes precipitation of an aluminium salt to which the TLR agonist and the immunogen are adsorbed. The invention also provides an immunogenic composition obtained or obtainable by this process.

In a thirteenth aspect, the invention provides a process for preparing a sterile immunogenic composition, comprising steps of combining (i) a S. aureus immunogen with (ii) a sterile complex of a TLR agonist and an insoluble metal salt. The sterile complex can be prepared by a process comprising steps of (a) mixing a TLR agonist and an insoluble metal salt such that the TLR agonist adsorbs to the insoluble metal salt to form the complex; and (b) sterilising the complex. Sterilisation can be conveniently achieved by autoclaving (or similar procedures [3]). As an alternative, the sterile complex can be prepared by (a) sterilising a solution or suspension of a TLR agonist and (b) combining the sterilised solution or suspension with a sterile insoluble metal salt; or by (a) sterilising an insoluble metal salt and (b) combining the sterilised insoluble metal salt with a sterile solution or suspension of a TLR agonist; or by combining (a) a sterile solution or suspension of a TLR agonist with (b) a sterile insoluble metal salt. Sterilisation of the TLR agonist solution/suspension can conveniently be achieved by sterile filtration, and this material can be prepared in concentrated form. Sterilisation of the insoluble metal salt can conveniently be achieved by autoclaving. The sterile insoluble metal salt will typically be an aqueous suspension.

In one embodiment, the invention provides an immunogenic composition comprising:
an aluminium hydroxide adjuvant;
a TLR7 agonist of formula (K), such as compound K2;
a first polypeptide comprising SEQ ID NO: 6, or a modified amino acid sequence which differs from SEQ ID NO: 6 by up to 5 single amino changes provided that the modified sequence can elicit antibodies which bind to a polypeptide consisting of SEQ ID NO: 6;
a second polypeptide comprising SEQ ID NO: 13, or a modified amino acid sequence which differs from SEQ ID NO: 13 by up to 5 single amino changes provided that the modified sequence can elicit antibodies which bind to a polypeptide consisting of SEQ ID NO: 13;
a third polypeptide comprising SEQ ID NO: 31, or a modified amino acid sequence which differs from SEQ ID NO: 31 by up to 5 single amino changes provided that the modified sequence can elicit antibodies which bind to a polypeptide consisting of SEQ ID NO: 31;
a fourth polypeptide comprising SEQ ID NO: 33, or a modified amino acid sequence which differs from SEQ ID NO: 33 by up to 5 single amino changes provided that the modified sequence can elicit antibodies which bind to a polypeptide consisting of SEQ ID NO: 33,
in which the TLR7 agonist and/or at least one of the polypeptides is/are adsorbed to the aluminium hydroxide adjuvant.

For example, as explained in more detail below: the first polypeptide can comprise SEQ ID NO: 41; the second polypeptide can comprise SEQ ID NO: 13; the third polypeptide can comprise SEQ ID NO: 47; and the fourth polypeptide can comprise SEQ ID NO: 43. Thus the composition can use a mixture of four polypeptides having SEQ ID NOs: 44, 27, 45 and 46.

### TLR Agonists

Compositions of the invention include a TLR agonist i.e. a compound which can agonise a Toll-like receptor. Most preferably, a TLR agonist is an agonist of a human TLR. The TLR agonist can activate any of TLR1, TLR2, TLR3, TLR4, TLR5, TLR6, TLR7, TLR8, TLR9 or TLR11; preferably it can activate human TLR7.

Agonist activity of a compound against any particular Toll-like receptor can be determined by standard assays. Companies such as Imgenex and Invivogen supply cell lines which are stably co-transfected with human TLR genes and NFκB, plus suitable reporter genes, for measuring TLR activation pathways. They are designed for sensitivity, broad working range dynamics and can be used for high-throughput screening. Constitutive expression of one or two specific TLRs is typical in such cell lines. See also reference 4. Many TLR agonists are known in the art e.g. reference 5 describes certain lipopeptide molecules that are TLR2 agonists, references 6 to 9 each describe classes of small molecule agonists of TLR7, and references 10 & 11 describe TLR7 and TLR8 agonists for treatment of diseases.

A TLR agonist used with the invention ideally includes at least one adsorptive moiety. The inclusion of such moieties in TLR agonists allows them to adsorb to insoluble metal salts (e.g. by ligand exchange or any other suitable mechanism) and improves their immunological behaviour (see reference 2). Phosphorus-containing adsorptive moieties are particularly useful, and so an adsorptive moiety may comprise a phosphate, a phosphonate, a phosphinate, a phosphonite, a phosphinite, etc.

Preferably the TLR agonist includes at least one phosphonate group.

Thus, in preferred embodiments, a composition of the invention includes a TLR7 agonist which includes a phosphonate group. This phosphonate group can allow adsorption of the agonist to an insoluble metal salt, such as to an aluminium salt.

TLR agonists useful with the invention may include a single adsorptive moiety, or may include more than one e.g. between 2 and 15 adsorptive moieties. Typically a compound will include 1, 2 or 3 adsorptive moieties.

Phosphorus-containing TLR agonists useful with the invention can be represented by formula (A1):

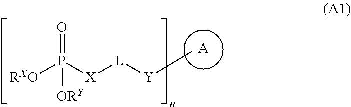

wherein:
   R^{X} and R^{Y} are independently selected from H and C₁-C₆ alkyl;
   X is selected from a covalent bond, O and NH;
   Y is selected from a covalent bond, O, C(O), S and NH;
   L is a linker e.g. selected from, C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
   each p is independently selected from 1, 2, 3, 4, 5 and 6;
   q is selected from 1, 2, 3 and 4;
   n is selected from 1, 2 and 3; and
   A is a TLR agonist moiety.

In one embodiment, the TLR agonist according to formula (A1) is as follows: R^{X} and R^{Y} are H; X is O; L is selected from C₁-C₆ alkylene and —((CH₂)ₚO)_{q}(CH₂)ₚ— each optionally substituted with 1 to 2 halogen atoms; p is selected from 1, 2 and 3; q is selected from 1 and 2; and n is 1. Thus in these embodiments the adsorptive moiety comprises a phosphate group.

In other embodiments, the TLR agonist according to formula (A1) is as follows: R^{X} and R^{Y} are H; X is a covalent bond; L is selected from C₁-C₆ alkylene and —((CH₂)ₚO)_{q}(CH₂)ₚ— each optionally substituted with 1 to 2 halogen atoms; p is selected from 1, 2 or 3; q is selected from 1 or 2; and n is 1. Thus in these embodiments the adsorptive moiety comprises a phosphonate group.

Useful ‘A’ moieties for formula (A1) include, but are not limited to, radicals of any of the following compounds, defined herein or as disclosed in references 4-11 and 34-52:

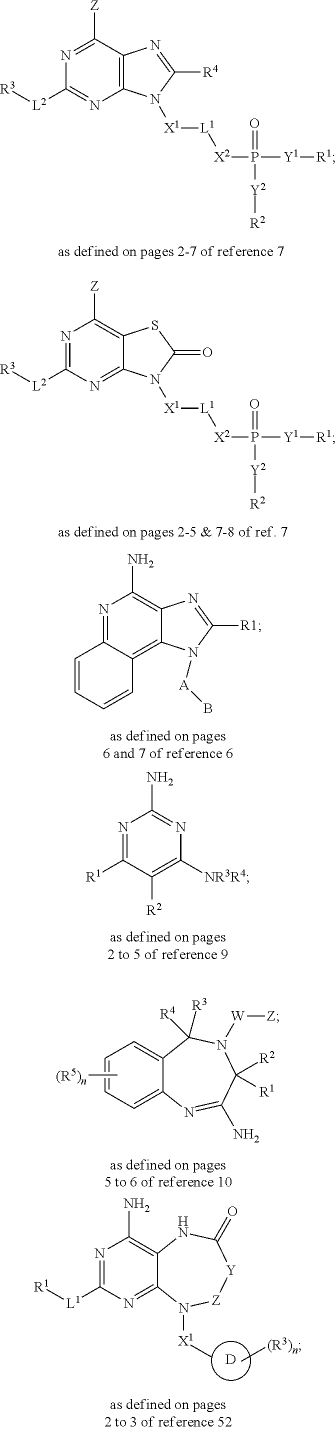

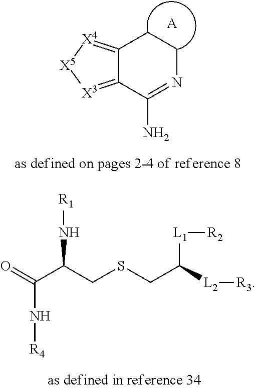

In some embodiments, the TLR agonist moiety ‘A’ has a molecular weight of less than 1000 Da. In some embodiments, the TLR agonist of formula (A1) has a molecular weight of less than 1000 Da.

Preferred TLR agonists are water-soluble. Thus they can form a homogenous solution when mixed in an aqueous buffer with water at pH 7 at 25° C. and 1 atmosphere pressure to give a solution which has a concentration of at least 50 μg/ml. The term “water-soluble” thus excludes substances that are only sparingly soluble under these conditions.

Useful TLR agonists include those having formula (C), (D), (E), (F), (G), (H), (I), (II), (J) or (K) as described in more detail below. Other useful TLR agonists are compounds 1 to 102 as defined in reference 2 (see pages 51-75 therein). Preferred TLR7 agonists have formula (K), such as ‘K2’. These can be used as salts e.g. the arginine salt of K2.

Preferred TLR4 agonists are analogs of monophosphoryl lipid A (MPL). For instance, a useful TLR4 agonist is a 3d-MPL (i.e. 3-O-deacylated monophosphoryl lipid A; also known as 3-de-O-acylated monophosphoryl lipid A or 3-O-desacyl-4′-monophosphoryl lipid A). The name indicates that position 3 of the reducing end glucosamine in monophosphoryl lipid A is de-acylated. It has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of cytokines, including IL-1, IL-12, TNF-α and GM-CSF. Preparation of 3d-MPL was originally described in reference 12, and the product has been manufactured and sold by Corixa Corporation. It is present in the AS04 adjuvant used by GlaxoSmithKline. Further details can be found in references 13 to 16. In some embodiments, however, the invention does not use a combination of aluminium phosphate and 3dMPL.

Typical compositions include 3d-MPL at a concentration of between 25 μg/ml and 200 μg/ml e.g. in the range 50-150 μg/ml, 75-125 μg/ml, 90-110 μg/ml, or about 100 μg/ml. It is usual to administer between 25-75 μg of 3d-MPL per dose e.g. between 45-55n, or about 50 μg 3d-MPL per dose.

3d-MPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}. The group attached to carbon 3′ has formula —O—CO—CH₂—CR³R^{3′}. A representative structure is:

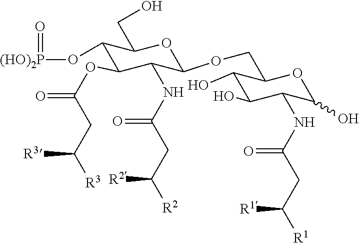

Groups R¹, R² and R³ are each independently —(CH₂)ₙ—CH₃. The value of n is preferably between 8 and 16, more preferably between 9 and 12, and is most preferably 10.

Groups R^{1′}, R^{2′} and R^{3′} can each independently be: (a) —H; (b) —OH; or (c) —O—CO—R⁴, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are —H then the 3d-MPL has only 3 acyl chains (one on each of positions 2, 2′ and 3′). When only two of R^{1′}, R^{2′} and R^{3′} are —H then the 3d-MPL can have 4 acyl chains. When only one of R^{1′}, R^{2′} and R^{3′} is —H then the 3d-MPL can have 5 acyl chains. When none of R^{1′}, R^{2′} and R^{3′} is —H then the 3d-MPL can have 6 acyl chains. The 3d-MPL used according to the invention can be a mixture of these forms, with from 3 to 6 acyl chains, but it is preferred to include 3d-MPL with 6 acyl chains in the mixture, and in particular to ensure that the 6 acyl chain form makes up at least 10% by weight of the total 3d-MPL e.g. ≧20%, ≧30%, ≧40%, ≧50% or more. 3d-MPL with 6 acyl chains has been found to be the most adjuvant-active form.

Thus the most preferred form of 3d-MPL for use with the invention is:

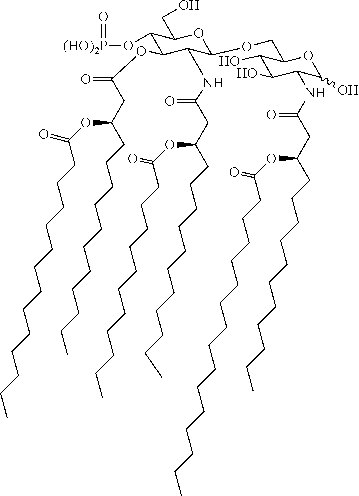

Where 3d-MPL is used in the form of a mixture then references to amounts or concentrations of 3d-MPL in compositions of the invention refer to the combined 3d-MPL species in the mixture.

In aqueous conditions, 3d-MPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3d-MPL) are preferred for use according to the invention because of their superior activity [17]. Preferred particles have a mean diameter less than 150 nm, more preferably less than 120 nm, and can even have a mean diameter less than 100 nm. In most cases, however, the mean diameter will not be lower than 50 nm. Where 3d-MPL is adsorbed to aluminum phosphate then it may not be possible to measure the 3D-MPL particle size directly, but particle size can be measured before adsorption takes place. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

A composition of the invention can include more than one TLR agonist. These two agonists are different from each other and they can target the same TLR or different TLRs. Both agonists can be adsorbed to a metal salt.

### Insoluble Metal Salts

TLR agonists can adsorb to insoluble metal salts to form an adsorbed complex for adjuvanting S. aureus antigens. For instance, they can be adsorbed to insoluble calcium salts (e.g. calcium phosphate) or, preferably, to insoluble aluminium salts. Such aluminium salts have a long history of use in vaccines.

Useful aluminium salts include, but are not limited to, aluminium hydroxide and aluminium phosphate adjuvants. Such salts are described e.g. in chapters 8 & 9 of reference 18, and chapter 4 of reference 19). Aluminium salts which include hydroxide ions are the preferred insoluble metal salts for use with the present invention as these hydroxide ions can readily undergo ligand exchange. Thus preferred salts for adsorption of TLR agonists are aluminium hydroxide and/or aluminium hydroxyphosphate. These have surface hydroxyl moieties which can readily undergo ligand exchange with phosphorus-containing groups (e.g. phosphates, phosphonates) to provide stable adsorption.

The adjuvants commonly known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 18). The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants e.g. with needle-like particles with diameters about 2 nm. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants commonly known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of reference 18).

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs, with primary particles in the range of 50 nm). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

In solution both aluminium phosphate and hydroxide adjuvants tend to form stable porous aggregates 1-10 μm in diameter [20].

A composition including an TLR agonist of the invention adsorbed to a metal salt can also include a buffer (e.g. a phosphate or a histidine or a Tris buffer). When such a composition includes a phosphate buffer, however, it is preferred that the concentration of phosphate ions in the buffer should be less than 50 mM e.g. <40 mM, <30 mM, <20 mM, <10 mM, or <5 mM, or between 1-15 mM. A histidine buffer is preferred e.g. between 1-50 mM, between 5-25 mM, or about 10 mM.

Because of the insolubility of adsorptive metal salts which are useful with the invention, compositions containing adsorbed immunopotentiators will generally be suspensions having a cloudy appearance. This can mask contaminating bacterial growth and so a composition of the invention may include a preservative such as thiomersal or 2-phenoxyethanol. It is preferred that a composition should be substantially free from (e.g. <10 μg/ml) mercurial material e.g. thiomersal-free. Vaccines containing no mercury are more preferred.

A composition can include a mixture of both an aluminium oxyhydroxide and an aluminium hydroxyphosphate, and a TLR agonist may be adsorbed to one or both of these salts.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range of Al⁺⁺⁺ in a composition of the invention is between 0.3 and 1 mg/ml or between 0.3-0.5 mg/ml. A maximum of 0.85 mg/dose is preferred. Because the inclusion of a TLR agonist can improve the adjuvant effect of aluminium salts then the invention advantageously permits lower amounts of Al⁺⁺⁺ per dose, and so a composition of the invention can usefully include between 10 and 250 μg of Al⁺⁺⁺ per unit dose. Current pediatric vaccines typically include at least 300 μg Al⁺⁺⁺. In concentration terms, a composition of the invention may have an Al⁺⁺⁺ concentration between 10 and 500 μg/ml e.g. between 10-300 μg/ml, between 10-200 μg/ml, or between 10-100 μg/ml.

In general, when a composition includes both a TLR agonist and an aluminium salt, the weight ratio of agonist to Al⁺⁺⁺ will be less than 5:1 e.g. less than 4:1, less than 3:1, less than 2:1, or less than 1:1. Thus, for example, with an Al⁺⁺⁺ concentration of 0.5 mg/ml the maximum concentration of TLR agonist would be 1.5 mg/ml. But higher or lower levels can be used.

Where a composition includes a TLR agonist and an insoluble metal salt, it is preferred that at least 50% (by mass) of the agonist in the composition is adsorbed to the metal salt e.g. ≧60%, ≧70%, ≧80%, ≧85%, ≧90%, ≧92%, ≧94%, ≧95%, ≧96%, ≧97%, ≧98%, ≧99%, or even 100%.

S. aureus Antigens

Compositions of the invention include either at least one S. aureus antigen or at least two S. aureus antigens. Thus a composition can include 1, 2, 3, 4, 5 or more S. aureus antigens; typically it will not include more than 10 different S. aureus antigens.

Both saccharide and polypeptide antigens are known for S. aureus (e.g. known saccharide antigens include the exopolysaccharide of S. aureus, which is a poly-N-acetylglucosamine (PNAG), and the capsular saccharides of S. aureus, which can be e.g. from type 5, type 8 or type 336). In preferred compositions the S. aureus antigen(s) is/are polypeptide antigen(s); in some embodiments a composition does not include a S. aureus saccharide antigen.

Preferred S. aureus polypeptide antigens for use with the invention are EsxA, EsxB, Sta006, Sta011, and/or Hla. These five antigens are discussed in detail in reference 1. A particularly useful composition of the invention includes all five of these antigens (preferably with a non-toxic mutant form of Hla).

The ‘EsxA’ antigen in the NCTC 8325 strain has amino acid sequence SEQ ID NO: 1 (GI:88194063). EsxA antigens used with the present invention can elicit an antibody (e.g. when administered to a human) that recognises SEQ ID NO: 1 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) comprising a fragment of at least ‘n’ consecutive amino acids of SEQ ID NO: 1, wherein ‘n’ is 7 or more (e.g. 8, 10, 12, 14, 16, 18, 20, 25, 30, 35, 40, 50, 60, 70, 80, 90 or more). These EsxA polypeptides include variants of SEQ ID NO: 1. Preferred fragments of (b) comprise an epitope from SEQ ID NO: 1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 1 while retaining at least one epitope of SEQ ID NO: 1.

The ‘EsxB’ antigen in the NCTC 8325 strain has amino acid sequence SEQ ID NO: 2 (GI:88194070). EsxB used with the present invention can elicit an antibody (e.g. when administered to a human) that recognises SEQ ID NO: 2 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) comprising a fragment of at least ‘n’ consecutive amino acids of SEQ ID NO: 2, wherein ‘n’ is 7 or more (e.g. 8, 10, 12, 14, 16, 18, 20, 25, 30, 35, 40, 50, 60, 70, 80, 90, 100 or more). These EsxB polypeptides include variants of SEQ ID NO: 2. Preferred fragments of (b) comprise an epitope from SEQ ID NO: 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 2 while retaining at least one epitope of SEQ ID NO: 2. A useful EsxB antigen lacks the internal cysteine residue of SEQ ID NO: 2 e.g. it comprises SEQ ID NO: 42, wherein residue X at position 30 is either absent or is an amino acid residue without a free thiol group (under reducing conditions) e.g. is any natural amino acid except cysteine.

The ‘Sta006’ antigen is annotated as ‘ferrichrome-binding protein’, and has also been referred to as ‘FhuD2’ in the literature [21]. In the NCTC 8325 strain Sta006 has amino acid sequence SEQ ID NO: 3 (GI:88196199). Sta006 used with the present invention can elicit an antibody (e.g. when administered to a human) that recognises SEQ ID NO: 3 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) comprising a fragment of at least ‘n’ consecutive amino acids of SEQ ID NO: 3, wherein ‘n’ is 7 or more (e.g. 8, 10, 12, 14, 16, 18, 20, 25, 30, 35, 40, 50, 60, 70, 80, 90, 100, 150, 200, 250 or more). These Sta006 polypeptides include variants of SEQ ID NO: 3. Preferred fragments of (b) comprise an epitope from SEQ ID NO: 3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 3 while retaining at least one epitope of SEQ ID NO: 3. The first 17 N-terminal amino acids of SEQ ID NO: 3 can usefully be omitted (to provide SEQ ID NO: 6). Mutant forms of Sta006 are reported in reference 22. A useful Sta006 antigen lacks the cysteine residue of SEQ ID NO: 3 e.g. it comprises SEQ ID NO: 41 and does not include any amino acid residue with a free thiol group (under reducing conditions) e.g. it is cysteine-free. A Sta006 antigen may be lipidated e.g. with an acylated N-terminus cysteine. One useful Sta006 sequence is SEQ ID NO: 7, which has a Met-Ala-Ser- sequence at the N-terminus; SEQ ID NO: 44 is another such sequence, but it lacks the cysteine present in SEQ ID NO: 7.

The ‘Sta011’ antigen has amino acid sequence SEQ ID NO: 4 (GI:88193872) in the NCTC 8325 strain. Sta011 antigens used with the invention can elicit an antibody (e.g. when administered to a human) that recognises SEQ ID NO: 4 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘n’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). These Sta011 polypeptides include variants of SEQ ID NO: 4. Preferred fragments of (b) comprise an epitope from SEQ ID NO: 4. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 4 while retaining at least one epitope of SEQ ID NO: 4. The first 23 N-terminal amino acids of SEQ ID NO: 4 can usefully be omitted (to provide SEQ ID NO: 33). A useful Sta011 antigen lacks the cysteine residue of SEQ ID NO: 4 e.g. it comprises SEQ ID NO: 43 and does not include any amino acid residue with a free thiol group (under reducing conditions) e.g. it is cysteine-free. A Sta011 antigen may be lipidated e.g. with an acylated N-terminus cysteine. One useful Sta011 sequence is SEQ ID NO: 8, which has a N-terminus methionine; SEQ ID NO: 46 is another such sequence, but it lacks the cysteine present in SEQ ID NO: 8. Variant forms of SEQ ID NO: 4 which may be used as or for preparing Sta011 antigens include, but are not limited to, SEQ ID NOs: 9, 10 and 11 with various Ile/Val/Leu substitutions (and Cys-free variants of these sequences can also be used with the invention). Sta011 can exist as a monomer or an oligomer, with Ca⁺⁺ ions favouring oligomerisation. The invention can use monomers and/or oligomers of Sta011.

The ‘Hla’ antigen is the ‘alpha-hemolysin precursor’ also known as ‘alpha toxin’ or simply ‘hemolysin’. In the NCTC 8325 strain Hla has amino acid sequence SEQ ID NO: 5 (GI:88194865). Hla is an important virulence determinant produced by most strains of S. aureus, having pore-forming and haemolytic activity. Anti-Hla antibodies can neutralise the detrimental effects of the toxin in animal models, and Hla is particularly useful for protecting against pneumonia.

Useful Hla antigens can elicit an antibody (e.g. when administered to a human) that recognises SEQ ID NO: 5 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). These Hla antigens include variants of SEQ ID NO: 5. Preferred fragments of (b) comprise an epitope from SEQ ID NO: 5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 5 while retaining at least one epitope of SEQ ID NO: 5. The first 26 N-terminal amino acids of SEQ ID NO: 5 can usefully be omitted (e.g. to give SEQ ID NO: 12). Truncation at the C-terminus can also be used e.g. leaving only 50 amino acids (residues 27-76 of SEQ ID NO: 5) [23].

Hla's toxicity can be avoided in compositions of the invention by chemical inactivation (e.g. using formaldehyde, glutaraldehyde or other cross-linking reagents). Instead, however, it is preferred to use mutant forms of Hla which remove its toxic activity while retaining its immunogenicity. Such detoxified mutants are already known in the art. A preferred Hla antigen is a mutant S. aureus hemolysin having a mutation at residue 61 of SEQ ID NO: 5, which is residue 35 of the mature antigen (i.e. after omitting the first 26 N-terminal amino acids=residue 35 of SEQ ID NO: 12). Thus residue 61 may not be histidine, and may instead be e.g. Ile, Val or preferably Leu. A His-Arg mutation at this position can also be used. For example, SEQ ID NO: 13 is the mature mutant Hla-H35L sequence (i.e. SEQ ID NO: 12 with a H35L mutation) and a useful Hla antigen comprises SEQ ID NO: 13. Another useful mutation replaces a long loop with a short sequence e.g. to replace the 39mer at residues 136-174 of SEQ ID NO: 5 with a tetramer such as PSGS (SEQ ID NO: 14), as in SEQ ID NO: 15 (which also includes the H35L mutation) and SEQ ID NO: 16 (which does not include the H35L mutation). Another useful mutation replaces residue Y101 e.g. with a leucine (SEQ ID NO: 17). Another useful mutation replaces residue D152 e.g. with a leucine (SEQ ID NO: 18). Another useful mutant replaces residues H35 and Y101 e.g. with a leucine (SEQ ID NO: 19). Another useful mutant replaces residues H35 and D152 e.g. with a leucine (SEQ ID NO: 20).

Further useful Hla antigens are disclosed in references 24 and 25.

SEQ ID NOs: 21, 22 & 23 are three useful fragments of SEQ ID NO: 5 (‘Hla₂₇₋₇₆’, ‘Hla₂₇₋₈₉’ and ‘Hla₂₇₋₇₉’, respectively). SEQ ID NOs: 24, 25 & 26 are the corresponding fragments from SEQ ID NO: 13.

One useful Hla sequence is SEQ ID NO: 27. It has a N-terminal Met, then an Ala-Ser dipeptide from the expression vector, then SEQ ID NO: 13 (from NCTC8325 strain).

Where a composition includes both EsxA and EsxB antigens, these may be present as a single polypeptide (i.e. as a fusion polypeptide). Thus a single polypeptide can elicit antibodies (e.g. when administered to a human) that recognise both SEQ ID NO: 1 and SEQ ID NO: 2. The single polypeptide can include: (i) a first polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1 and/or comprising a fragment of at least ‘n’ consecutive amino acids of SEQ ID NO: 1, as defined above for EsxA; and (ii) a second polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2 and/or comprising a fragment of at least ‘n’ consecutive amino acids of SEQ ID NO: 2, as defined above for EsxB. The first and second polypeptide sequences can be in either order, N- to C-terminus. SEQ ID NOs: 28 (‘EsxAB’) and 29 (‘EsxBA’) are examples of such polypeptides, both having hexapeptide linkers ASGGGS (SEQ ID NO: 30). Another ‘EsxAB’ hybrid comprises SEQ ID NO: 31, which may be provided with a N-terminus methionine (e.g. SEQ ID NO: 32). A useful variant of EsxAB lacks the internal cysteine residue of EsxB e.g. it comprises SEQ ID NO: 47 wherein residue X at position 132 is either absent or is an amino acid residue without a free thiol group (under reducing conditions) e.g. is any natural amino acid except cysteine. Thus a preferred EsxAB antigen for use with the invention has amino acid sequence SEQ ID NO: 45.

Thus a useful polypeptide comprises an amino acid sequence (a) having 80% or more identity (e.g. 80%, 85%, 90%, 91%, 92%, 93%, 94%, 95%, 96%, 97%, 98%, 99%, 99.5% or more) to SEQ ID NO: 31; and/or (b) comprising both a fragment of at least ‘n’ consecutive amino acids from amino acids 1-96 of SEQ ID NO: 31 and a fragment of at least ‘n’ consecutive amino acids from amino acids 103-205 of SEQ ID NO: 31, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides (e.g. SEQ ID NO: 32) can elicit antibodies (e.g. when administered to a human) which recognise both the wild-type staphylococcal protein comprising SEQ ID NO: 1 and the wild-type staphylococcal protein comprising SEQ ID NO: 2. Thus the immune response will recognise both of antigens esxA and esxB. Preferred fragments of (b) provide an epitope from SEQ ID NO: 1 and an epitope from SEQ ID NO: 2.

A preferred composition of the invention thus includes all four of: (i) a single polypeptide including both an EsxA antigen and an EsxB antigen e.g. comprising SEQ ID NO: 31; (ii) a Sta006 antigen e.g. comprising SEQ ID NO: 6; (iii) a Sta011 antigen e.g. comprising SEQ ID NO: 33; and (iv) a H35L mutant form of Hla e.g. comprising SEQ ID NO: 13. This composition is particularly useful when using TLR7 agonists of formula (K).

Although SEQ ID NOs: 31, 6, 33 and 13 are useful amino acid sequences in a combination, the invention is not limited to these precise sequences. Thus 1, 2, 3 or all 4 of these sequences can independently be modified by up to 5 single amino changes (i.e. 1, 2, 3, 4 or 5 single amino acid substitutions, deletions and/or insertions) provided that the modified sequence can elicit antibodies which still bind to a polypeptide consisting of the unmodified sequence.

One useful composition of the invention includes all four of: (i) a first polypeptide having amino acid sequence SEQ ID NO: 32; (ii) a second polypeptide having amino acid sequence SEQ ID NO: 7; (iii) a third polypeptide having amino acid sequence SEQ ID NO: 8; and (iv) a fourth polypeptide having amino acid sequence SEQ ID NO: 27. Again, this composition is particularly useful when using TLR7 agonists of formula (K). In some embodiments the composition may include one or more further polypeptides; in other embodiments the only polypeptides in the composition are these four specified polypeptides. SEQ ID NOs: 32, 7, 8 and 27 are useful amino acid sequences in a combination, but the invention is not limited to these precise sequences. Thus 1, 2, 3 or all 4 of these four sequences can independently be modified by 1, 2, 3, 4 or 5 single amino changes (i.e. 1, 2, 3, 4 or 5 single amino acid substitutions, deletions and/or insertions) provided that the modified sequence can elicit antibodies which still bind to a polypeptide consisting of the unmodified sequence. In a preferred embodiment, the composition thus includes these four specified polypeptides with 1, 2, 3 or all 4 of SEQ ID NO: 32, 7, 8 and 27 independently modified by 1 single amino acid substitution, deletion and/or insertion.

For instance, wild-type Sta006, Sta011 and EsxAB polypeptide sequences (e.g. SEQ ID NOs: 6, 31 and 33) each include a single cysteine residue which can lead to inter-polypeptide disulfide bridges, forming both homodimers and heterodimers. Such inter-linked polypeptides are undesirable and so Sta006, Sta011 and EsxB sequences can be modified to remove their natural cysteine residues, such that they do not contain free thiol groups (under reducing conditions). The wild-type cysteine can be deleted or can be substituted with a different amino acid.

Thus: a Sta006 antigen can comprise SEQ ID NO: 41; a Sta011 antigen can comprise SEQ ID NO: 43; and a EsxB antigen can comprise SEQ ID NO: 42 (e.g. as an EsxAB hybrid comprising SEQ ID NO: 47). Examples of such sequences include, but are not limited to, SEQ ID NOs: 44, 46, and 45. These sequences can be used singly as substitutes for the corresponding wild-type sequences, or in combination. Thus a particularly useful composition of the invention includes all four of: (i) a first polypeptide having amino acid sequence SEQ ID NO: 45; (ii) a second polypeptide having amino acid sequence SEQ ID NO: 44; (iii) a third polypeptide having amino acid sequence SEQ ID NO: 46; and (iv) a fourth polypeptide having amino acid sequence SEQ ID NO: 27. In some embodiments the composition may include one or more further polypeptides; in other embodiments the only polypeptides in the composition are these four specified polypeptides. This combination of polypeptides is particularly useful when using TLR7 agonists of formula (K), such as of formula K2 e.g. in conjunction with adsorption of the agonist and/or polypeptides to an aluminium hydroxide adjuvant, as discussed above.

Other compositions of the invention, particularly when using 3d-MPL as an adsorbed TLR agonist (e.g. adsorbed to an aluminium salt), can include a ClfA antigen, an IsdA antigen, an IsdB antigen, an IsdC antigen, and/or an IsdH antigen.

The ‘ClfA’ antigen, or ‘clumping factor A’, in the NCTC 8325 strain has amino acid sequence SEQ ID NO: 34 (GI:88194572). ClfA antigens used with the present invention can elicit an antibody (e.g. when administered to a human) that recognises SEQ ID NO: 34 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) comprising a fragment of at least ‘n’ consecutive amino acids of SEQ ID NO: 34, wherein ‘n’ is 7 or more (e.g. 8, 10, 12, 14, 16, 18, 20, 25, 30, 35, 40, 50, 60, 70, 80, 90, 100, 150, 200, 250 or more). These ClfA antigens include variants of SEQ ID NO: 34. Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34 while retaining at least one epitope of SEQ ID NO: 34. The final 368 C-terminal amino acids of SEQ ID NO: 34 can usefully be omitted. The first 39 N-terminal amino acids of SEQ ID NO: 34 can usefully be omitted. SEQ ID NO: 40 is a useful fragment of SEQ ID NO: 34 (‘ClfA₄₀₋₅₅₉’), which omits the long repetitive region towards the C-terminal of SEQ ID NO: 34. ClfA antigens used with the invention can usefully be modified from wild-type sequences to reduce or remove their affinity for fibrinogen e.g. the Y474 mutation of reference 26, the D321 mutation of reference 27, etc.

The ‘IsdA’ antigen in the NCTC 8325 strain has amino acid sequence SEQ ID NO: 35 (GI:88194829). Anti-IsdA antibodies can protect mice against S. aureus abscess formation and lethal challenge [28]. IsdA antigens used with the invention can elicit an antibody (e.g. when administered to a human) that recognises SEQ ID NO: 35 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 35; and/or (b) comprising a fragment of at least ‘n’ consecutive amino acids of SEQ ID NO: 35, wherein ‘n’ is 7 or more (e.g. 8, 10, 12, 14, 16, 18, 20, 25, 30, 35, 40, 50, 60, 70, 80, 90, 100, 150, 200, 250 or more). These IsdA antigens include variants of SEQ ID NO: 35. Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 35 while retaining at least one epitope of SEQ ID NO: 35. The final 38 C-terminal amino acids of SEQ ID NO: 35 can usefully be omitted. The first 46 N-terminal amino acids of SEQ ID NO: 35 can usefully be omitted. Truncation to exclude the C-terminal 38mer of SEQ ID NO: 35 (beginning with the LPKTG motif) is also useful. SEQ ID NO: 36 is a useful fragment of SEQ ID NO: 35 (amino acids 40-184 of SEQ ID NO: 35; ‘IsdA₄₀₋₁₈₄’) which includes the natural protein's heme binding site and includes the antigen's most exposed domain. It also reduces the antigen's similarity with human proteins. Other useful fragments are disclosed in references 29 and 30.

The ‘IsdB’ antigen in the NCTC 8325 strain has amino acid sequence SEQ ID NO: 37 (GI:88194828). Anti-IsdB antibodies can protect mice against S. aureus abscess formation and lethal challenge [28]. IsdB antigens used with the present invention can elicit an antibody (e.g. when administered to a human) that recognises SEQ ID NO: 37 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) comprising a fragment of at least ‘n’ consecutive amino acids of SEQ ID NO: 37, wherein ‘n’ is 7 or more (e.g. 8, 10, 12, 14, 16, 18, 20, 25, 30, 35, 40, 50, 60, 70, 80, 90, 100, 150, 200, 250 or more). These IsdB antigens include variants of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37 while retaining at least one epitope of SEQ ID NO: 37. The final 36 C-terminal amino acids of SEQ ID NO: 37 can usefully be omitted. The first 40 N-terminal amino acids of SEQ ID NO: 37 can usefully be omitted. Useful fragments of IsdB are disclosed in references 30 and 31 e.g. lacking 37 internal amino acids of SEQ ID NO: 37.

The ‘IsdC’ antigen in the NCTC 8325 strain has amino acid sequence SEQ ID NO: 38 (GI:88194830). IsdC antigens used with the present invention can elicit an antibody (e.g. when administered to a human) that recognises SEQ ID NO: 38 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 38; and/or (b) comprising a fragment of at least ‘n’ consecutive amino acids of SEQ ID NO: 38, wherein ‘n’ is 7 or more (e.g. 8, 10, 12, 14, 16, 18, 20, 25, 30, 35, 40, 50, 60, 70, 80, 90, 100, 150, 200 or more). These IsdC antigens include variants of SEQ ID NO: 38. Preferred fragments of (b) comprise an epitope from SEQ ID NO: 38. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 38 while retaining at least one epitope of SEQ ID NO: 38. The final 39 C-terminal amino acids of SEQ ID NO: 38 can usefully be omitted. The first 28 N-terminal amino acids of SEQ ID NO: 38 can usefully be omitted.

The ‘IsdH’ antigen, also known as ‘HarA’, in the NCTC 8325 strain has amino acid sequence SEQ ID NO: 39 (GI:88195542). IsdH antigens used with the present invention can elicit an antibody (e.g. when administered to a human) that recognises SEQ ID NO: 39 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 39; and/or (b) comprising a fragment of at least ‘n’ consecutive amino acids of SEQ ID NO: 39, wherein ‘n’ is 7 or more (e.g. 8, 10, 12, 14, 16, 18, 20, 25, 30, 35, 40, 50, 60, 70, 80, 90, 100, 150, 200, 250 or more). These isdH antigens include variants of SEQ ID NO: 39. Preferred fragments of (b) comprise an epitope from SEQ ID NO: 39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 39 while retaining at least one epitope of SEQ ID NO: 39. The final 35 C-terminal amino acids of SEQ ID NO: 39 can usefully be omitted. The first 40 N-terminal amino acids of SEQ ID NO: 39 can usefully be omitted.

When using IsdA, IsdB, IsdC and/or IsdH it can be helpful to use a fusion polypeptide comprising epitopes from more than one of IsdA, IsdB, IsdC and/or IsdH. For instance, reference 32 discloses polypeptides which usefully include epitopes from both IsdB and IsdH. Similarly, reference 33 discloses polypeptides which usefully include epitopes from both IsdA and IsdB, and also some polypeptides which include epitopes from IsdA, IsdB and IsdH. When making these fusion polypeptides it can be helpful to include a NEAT domain from each polypeptide [33].

In some embodiments, a composition of the invention includes a S. aureus antigen and also an antigen from a different organism (e.g. from a virus or from another bacterium).

In some embodiments, the invention does not encompass compositions which include a combination of an IsdA antigen, an IsdB antigen, a ClfA antigen, a ClfB antigen, a SdrD antigen, a Spa antigen, an EsxA antigen, an EsxB antigen, a Sta006 antigen, a hemolysin, and a Sta011 antigen.

### Formulae (C), (D), (E) and (H)— TLR7 Agonists

The TLR agonist can be a compound according to any of formulae (C), (D), (E), and (H):

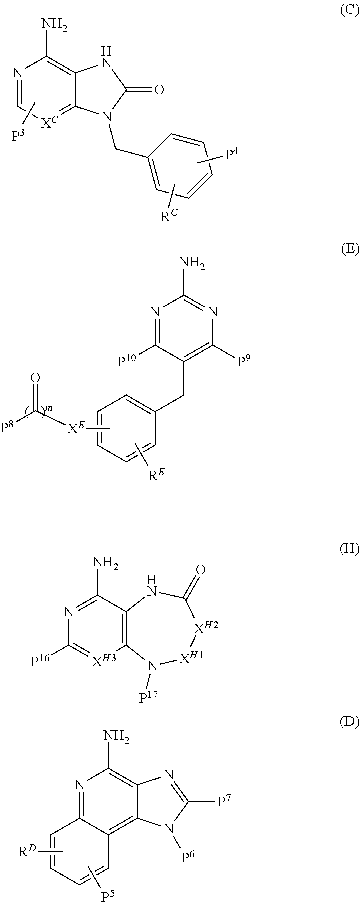

wherein:
(a) P³ is selected from H, C₁-C₆alkyl, CF₃, and —((CH₂)ₚO)_{q}(CH₂)ₚOₛ— and —Y-L-X—P(O)(OR^{X})(OR^{Y}); and P⁴ is selected from H, C₁-C₆alkyl, —C₁-C₆alkylaryl and —Y-L-X—P(O)(OR^{X})(OR^{Y}); with the proviso that at least one of P³ and P⁴ is —Y-L-X—P(O)(OR^{X})(OR^{Y}),
(b) P⁵ is selected from H, C₁-C₆alkyl, and —Y-L-X—P(O)(OR^{X})(OR^{Y}); P⁶ is selected from H, C₁-C₆alkyl each optionally substituted with 1 to 3 substituents selected from C₁-C₄alkyl and OH, and —Y-L-X—P(O)(OR^{X})(OR^{Y}); and P⁷ is selected from H, C₁-C₆alkyl, —((CH₂)ₚO)_{q}(CH₂)ₚOₛ—, —NHC₁-C₆alkyl and —Y-L-X—P(O)(OR^{X})(OR^{Y}); with the proviso that at least one of P⁵, P⁶ and P⁷ is —Y-L-X—P(O)(OR^{X})(OR^{Y});
(c) P⁸ is selected from H, C₁-C₆alkyl, C₁-C₆alkoxy, —NHC₁-C₆alkyl each optionally substituted with OH, and —Y-L-X—P(O)(OR^{X})(OR^{Y}); and P⁹ and P¹⁰ are each independently selected from H, C₁-C₆alkyl, C₁-C₆alkoxy, —NHC₁-C₆alkyl each optionally substituted with OH and C₁-C₆alkyl, and —Y-L-X—P(O)(OR^{X})(OR^{Y}); with the proviso that at least one of P⁸, P⁹ or P¹⁰ is —Y-L-X—P(O)(OR^{X})(OR^{Y});
(d) P¹⁶ and each P¹⁸ are each independently selected from H, C₁-C₆alkyl, and —Y-L-X—P(O)(OR^{X})(OR^{Y}); P¹⁷ is selected from H, C₁-C₆alkyl, aryl, heteroaryl, C₁-C₆alkylaryl, C₁-C₆alkyl heteroaryl, C₁-C₆alkylaryl-Y-L-X—P(O)(OR^{X})(OR^{Y}) and —Y-L-X—P(O)(OR^{X})(OR^{Y}), each optionally substituted with 1 to 2 substituents selected from C₁-C₆alkyl or heterocyclyl with the proviso that at least one of P¹⁶, P¹⁷ or a P¹⁸ contains a —Y-L-X—P(O)(OR^{X})(OR^{Y}) moiety;
R^{X} and R^{Y} are independently selected from H and C₁-C₆alkyl;
R^{C}, R^{D} and R^{H} are each independently selected from H and C₁-C₆alkyl;
X^{C} is selected from CH and N;
R^{E} is selected from H, C₁-C₆alkyl, C₁-C₆alkoxy, C(O)C₁-C₆alkyl, halogen and —((CH₂)ₚO)_{q}(CH₂)ₚ—;
X^{E} is selected from a covalent bond, CR^{E2}R^{E3} and NR^{E4};
R^{E2}, R^{E3} and R^{E4} are independently selected from H and C₁-C₆alkyl;
X^{H1}—X^{H2} is selected from —CR^{H2}R^{H3}—, —CR^{H2}R^{H3}—CR^{H2}R^{H3}—, —C(O)CR^{H2}R^{H3}—, —C(O)CR^{H2}R^{H3}—, —CR^{H2}R^{H3}C(O)—, —NR^{H4}C(O)—, C(O)NR^{H4}—, CR^{H2}R^{H3}S(O)₂ and —CR^{H2}═CR^{H2}—;
R^{H2}, R^{H3} and R^{H4} are each independently selected from H, C₁-C₆alkyl and P¹⁸;
X^{H3} is selected from N and CN;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
m is selected from 0 or 1;
each p is independently selected from 1, 2, 3, 4, 5 and 6;
q is selected from 1, 2, 3 and 4; and
s is selected from 0 and 1.

### Formula (G)—TLR⁸ Agonist

The TLR agonist can be a compound according to formula (G):

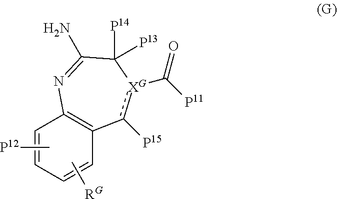

wherein:
P¹¹ is selected from H, C₁-C₆alkyl, C₁-C₆ alkoxy, NR^{V}R^{W} and —Y-L-X—P(O)(OR^{X})(OR^{Y});
P¹² is selected from H, C₁-C₆alkyl, aryl optionally substituted by —C(O)NR^{V}R^{W}, and —Y-L-X—P(O)(OR^{X})(OR^{Y});
P¹³, P¹⁴ and P¹⁵ are independently selected from H, C₁-C₆alkyl, C₁-C₆ alkoxy and —Y-L-X—P(O)(OR^{X})(OR^{Y});
with the proviso that at least one of P¹¹, P¹², P¹³, P¹⁴ or P¹⁵ is —Y-L-X—P(O)(OR^{X})(OR^{Y});
R^{V} and R^{W} are independently selected from H, C₁-C₆alkyl or together with the nitrogen atom to which they are attached form a 4 to 7 remembered heterocyclic ring;
X^{G} is selected from C, CH and N;
[symbol] represents an optional double bond, wherein X^{G} is C if [symbol] is a double bond; and
R^{G} is selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6 and
q is selected from 1, 2, 3 and 4.

### Formulae (I) and (II) — TLR⁷ Agonists [7]

The TLR agonist can be a compound according to formula (I) or formula (II):

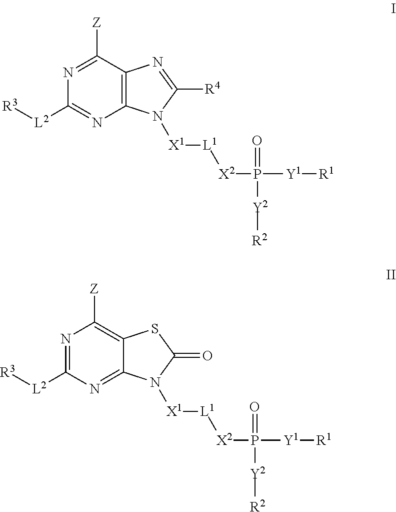

wherein:
   Z is —NH₂ or —OH;
   X¹ is alkylene, substituted alkylene, alkenylene, substituted alkenylene, alkynylene, substituted alkynylene, carbocyclylene, substituted carbocyclylene, heterocyclylene, or substituted heterocyclylene;
   L¹ is a covalent bond, arylene, substituted arylene, heterocyclylene, substituted heterocyclylene, carbocyclylene, substituted carbocyclylene, —S—, —S(O)—, S(O)₂, —NR⁵—, or —O—
   X² is a covalent bond, alkylene, or substituted alkylene;
   L² is NR⁵—, —N(R⁵)C(O)—, —O—, —S—, —S(O)—, S(O)₂, or a covalent bond;
   R³ is H, alkyl, substituted alkyl, heteroalkyl, substituted heteroalkyl, alkenyl, substituted alkenyl, aryl, substituted aryl, arylalkyl, substituted arylalkyl, heterocyclyl, substituted heterocyclyl, heterocyclylalkyl, or substituted heterocyclylalkyl;
   Y¹ and Y² are each independently a covalent bond, —O— or —NR⁵—; or —Y—R¹ and —Y²—R² are each independently —O—N═C(R⁶R⁷);
   R¹ and R² are each independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, substituted heterocyclylalkyl, -alkylene-C(O)—O—R⁵, -(substituted alkylene)-C(O)—O—R⁵, -alkylene-O—C(O)—R⁵, -(substituted alkylene)-O—C(O)—R⁵, -alkylene-O—C(O)—O—R⁵, or -(substituted alkylene)-O—C(O)—O—R⁵
   R⁴ is H, halogen, —OH, —O-alkyl, —O-alkylene-O—C(O)—O—R⁵, —O—C(O)—O—R⁵, —SH, or —NH(R⁵);
   each R⁵, R⁶, and R⁷ are independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, or substituted heterocyclylalkyl.

### Formula (J)—TLR² Agonists [34]

The TLR agonist can be a compound according to formula (J):

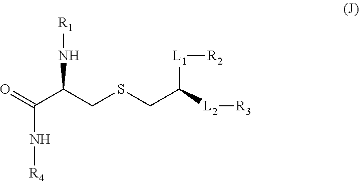

wherein:
R¹ is H, —C(O)—C₇-C₁₈alkyl or —C(O)—C₁-C₆alkyl;
R² is C₇-C₁₈alkyl;
R³ is C₇-C₁₈alkyl;
L₁ is —CH₂OC(O)—, —CH₂O—, —CH₂NR⁷C(O)— or —CH₂OC(O)NR⁷—;
L₂ is —OC(O)—, —O—, —NR⁷C(O)— or —OC(O)NR⁷—;
R⁴ is -L₃R⁵ or -L⁴R⁵;
R⁵ is —N(R⁷)₂, —OR⁷, —P(O)(OR⁷)₂, —C(O)OR⁷, —NR⁷C(O)L₃R⁸, —NR⁷C(O)L₄R⁸, —OL₃R⁶, —C(O)NR⁷L₃R⁸, —C(O)NR⁷L₄R⁸, —S(O)₂OR⁷, —OS(O)₂OR⁷, C₁-C₆alkyl, a C₆aryl, a C₁₀aryl, a C₁₄aryl, 5 to 14 ring membered heteroaryl containing 1 to 3 heteroatoms selected from O, S and N, C₃-C₈cycloalkyl or a 5 to 6 ring membered heterocycloalkyl containing 1 to 3 heteroatoms selected from O, S and N, wherein the aryl, heteroaryl, cycloalkyl and heterocycloalkyl of R⁵ are each unsubstituted or the aryl, heteroaryl, cycloalkyl and heterocycloalkyl of R⁵ are each substituted with 1 to 3 substituents independently selected from —OR⁹, —OL₃R⁶, —OL₄R⁶, —OR⁷, and —C(O)OR⁷;
L₃ is a C₁-C₁₀alkylene, wherein the C₁-C₁₀alkylene of L₃ is unsubstituted, or the C₁-C₁₀alkylene of L₃ is substituted with 1 to 4 R⁶ groups, or the C₁-C₁₀alkylene of L₃ is substituted with 2 C₁-C₆alkyl groups on the same carbon atom which together, along with the carbon atom they are attached to, form a C₃-C₈cycloakyl;
L₄ is —((CR⁷R⁷)ₚO)_{q}(CR¹⁰R¹⁰)ₚ— or —(CR¹¹R¹¹)((CR⁷R⁷)ₚO)_{q}(CR¹⁰R¹⁰)ₚ—, wherein each R¹¹ is a C₁-C₆alkyl groups which together, along with the carbon atom they are attached to, form a C₃-C₈cycloakyl;
each R⁶ is independently selected from halo, C₁-C₆alkyl, C₁-C₆alkyl substituted with 1-2 hydroxyl groups, —OR⁷, —N(R⁷)₂, —C(O)OH, —C(O)N(R⁷)₂, —P(O)(OR⁷)₂, a C₆aryl, a C₁₀aryl and a C₁₄aryl;
each R⁷ is independently selected from H and C₁-C₆alkyl;
R⁸ is selected from —SR⁷, —C(O)OH, —P(O)(OR⁷)₂, and a 5 to 6 ring membered heterocycloalkyl containing 1 to 3 heteroatoms selected from O and N;
R⁹ is phenyl;
each R¹⁰ is independently selected from H and halo;
each p is independently selected from 1, 2, 3, 4, 5 and 6, and
q is 1, 2, 3 or 4.

Preferably R⁵ is P(O)(OR⁷)₂, —NR⁷C(O)L₃-P(O)(OR⁷)₂, —NR⁷C(O)L₄-P(O)(OR⁷)₂, —OL₃-P(O)(OR⁷)₂, —C(O)NR⁷L³-P(O)(OR⁷)₂, or —C(O)NR⁷L⁴-P(O)(OR⁷)₂.

In some embodiments of (J), R₁ is H. In other embodiments of (J), R₁ is —C(O)—C₁₅alkyl;

In some embodiments of (J): (i) L₁ is —CH₂OC(O)— and L₂ is —OC(O)—, —O—, —NR⁷C(O)— or —OC(O)NR⁷—; or (ii) or L₁ is —CH₂O— and L₂ is —OC(O)—, —O—, —NR⁷C(O)— or —OC(O)NR⁷—; or (iii) L₁ is —CH₂NR⁷C(O)— and L₂ is —OC(O)—, —O—, —NR⁷C(O)— or —OC(O)NR⁷—; or (iv) L₁ is —CH₂OC(O)NR⁷— and L₂ is —OC(O)—, —O—, NR⁷C(O)— or —OC(O)NR⁷—.

In some embodiments of (J): (i) L₁ is —CH₂OC(O)— and L₂ is —OC(O)—; or (ii) L₁ is —CH₂O— and L₂ is —O—; or (iii) L₁ is —CH₂O— and L₂ is —NHC(O)—; or (iv) L₁ is —CH₂OC(O)NH— and L₂ is —OC(O)NH—.

In some embodiments of (J), (i) R² is —C₁₁alkyl and R³ is —C₁₁alkyl; or (ii) R² is —C₁₆alkyl and R³ is —C₁₆alkyl; or (iii) R² is —C₁₆alkyl and R³ is —C₁₁alkyl; or (iv) R² is —C₁₂alkyl and R³ is —C₁₂alkyl; or (v) R² is —C₇alkyl and R³ is —C₇alkyl; or (vi) R² is —C₉alkyl and R³ is —C₉alkyl; or (vii) R² is —C₈alkyl and R³ is —C₈alkyl; or (viii) R² is —C₁₃alkyl and R³ is —C₁₃alkyl; or (ix) R² is —C₁₂alkyl and R³ is —C₁₁alkyl; or (x) R² is —C₁₂alkyl and R³ is —C₁₂alkyl; or (xi) R² is —C₁₀alkyl and R³ is —C₁₀alkyl; or (xii) R² is —C₁₅alkyl and R³ is —C₁₅alkyl.

In some embodiments of (J), R² is —C₁₁alkyl and R³ is —C₁₁alkyl.

In some embodiments of (J), L₃ is a C₁-C₁₀alkylene, wherein the C₁-C₁₀alkylene of L₃ is unsubstituted or is substituted with 1 to 4 R⁶ groups.

In some embodiments of (J): L₄ is —((CR⁷R⁷)ₚO)_{q}(CR¹⁰R¹⁰)ₚ—; each R¹⁰ is independently selected from H and F; and each p is independently selected from 2, 3, and 4.

In some embodiments of (J), each R⁶ is independently selected from methyl, ethyl, i-propyl, i-butyl, —CH₂OH, —OH, —F, —NH₂, —C(O)OH, —C(O)NH₂, —P(O)(OH)₂ and phenyl.

In some embodiments of (J), each R⁷ is independently selected from H, methyl and ethyl.

### Formula (K) [35]

The TLR agonist can be a compound according to formula (K):

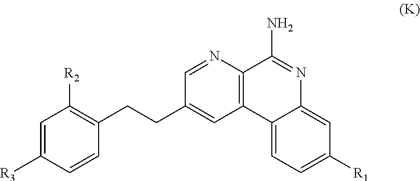

wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl;
R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₂P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

The compound of formula (K) is preferably of formula (K′):

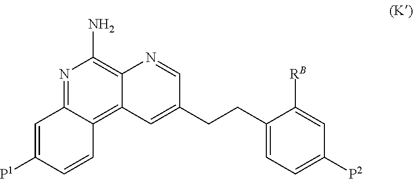

wherein:
P¹ is selected from H, C₁-C₆alkyl optionally substituted with COOH and —Y-L-X—P(O)(OR^{X})(OR^{Y});
P² is selected from H, C₁-C₆alkyl, C₁-C₆alkoxy and —Y-L-X—P(O)(OR^{X})(OR^{Y});
with the proviso that at least one of P¹ and P² is —Y-L-X—P(O)(OR^{X})(OR^{Y});
R^{B} is selected from H and C₁-C₆alkyl;
R^{X} and R^{Y} are independently selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6; and
q is selected from 1, 2, 3 and 4.

In some embodiments of formula (K′): P¹ is selected from C₁-C₆alkyl optionally substituted with COOH and —Y-L-X—P(O)(OR^{X})(OR^{Y}); P² is selected from C₁-C₆alkoxy and —Y-L-X—P(O)(OR^{X})(OR^{Y}); R^{B} is C₁-C₆alkyl; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

Formula (F)—TLR7 agonists [8]

The TLR agonist can be a compound according to formula (F):

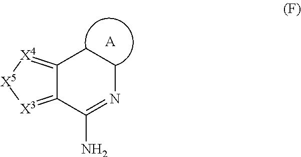

wherein:
   X³ is N;
   X⁴ is N or CR³
   X⁵ is —CR⁴═CR⁵—;
   R¹ and R² are H;
   R³ is H;
   R⁴ and R⁵ are each independently selected from H, halogen, —C(O)OR⁷, —C(O)R⁷, —C(O)N(R¹¹R¹²), —N(R¹¹R¹²), —N(R⁹)₂, —NHN(R⁹)₂, —SR⁷, —(CH₂)ₙOR⁷, —(CH₂)ₙR⁷, -LR⁸, -LR¹⁰, —OLR⁸, —OLR¹⁰, C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl groups of R⁴ and R⁵ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂. —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
   or, R³ and R⁴, or R⁴ and R⁵, or R⁵ and R⁶, when present on adjacent ring atoms, can optionally be linked together to form a 5-6 membered ring, wherein the 5-6 membered ring is optionally substituted with R⁷;
   each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂, —P(O)(OR¹⁰)₂, and —OP(O)(OR¹⁰)₂;
   R⁷ is selected from H, C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl, wherein the C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl groups of R⁷ are each optionally substituted with 1 to 3 R¹³ groups, and each R¹³ is independently selected from halogen, —CN, -LR⁹, -LOR⁹, —OLR⁹, -LR¹⁰, -LOR¹⁰, —OLR¹⁰, -LR⁸, -LOR⁸, —OLR⁸, -LSR⁸, -LSR¹⁰, -LC(O)R⁸, —OLC(O)R⁸, -LC(O)OR⁸, -LC(O)R¹⁰, -LOC(O)OR⁸, -LC(O)NR⁹R¹¹, -LC(O)NR⁹R⁸, -LN(R⁹)₂, -LNR⁹R⁸, -LNR⁹R¹⁰, -LC(O)N(R⁹)₂, -LS(O)₂R⁸, -LS(O)R⁸, -LC(O)NR⁸OH, -LNR⁹C(O)R⁸, -LNR⁹C(O)OR⁸, -LS(O)₂N(R⁹)₂, —OLS(O)₂N(R⁹)₂, -LNR⁹S(O)₂R⁸, -LC(O)NR⁹LN(R⁹)₂, -LP(O)(OR⁸)₂, -LOP(O)(OR⁸)₂, -LP(O)(OR¹⁰)₂ and —OLP(O)(OR¹⁰)₂;
   each R⁸ is independently selected from H, —CH(R¹⁰)₂, C₁-C₈alkyl, C₂-C₈alkene, C₂-C₈alkyne, C₁-C₆haloalkyl, C₁-C₆alkoxy, C₁-C₆heteroalkyl, C₃-C₈cycloalkyl, C₂-C₈heterocycloalkyl, C₁-C₆hydroxyalkyl and C₁-C₆haloalkoxy, wherein the C₁-C₈alkyl, C₂-C₈alkene, C₂-C₈alkyne, C₁-C₆heteroalkyl, C₁-C₆haloalkyl, C₁-C₆alkoxy, C₃-C₈cycloalkyl, C₂-C₈heterocycloalkyl, C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹, —C(O)N(R⁹)₂, —C(O)OR¹¹, —NR⁹C(O)R¹¹, —NR⁹R¹⁰, —NR¹¹R¹²—N(R⁹)₂, —OR⁹, —OR¹⁰, —C(O)NR¹¹R¹², —C(O)NR¹¹OH, —S(O)₂R¹¹, —S(O)R¹¹, —S(O)₂NR¹¹R¹²NR¹¹S(O)₂R¹¹, —P(O)(OR¹¹)₂, and —OP(O)(OR¹¹)₂;
   each R⁹ is independently selected from H, —C(O)R⁸, —C(O)OR⁸, —C(O)R¹⁰, —C(O)OR¹⁰, —S(O)₂R¹⁰, —C₁-C₆ alkyl, C₁-C₆ heteroalkyl and C₃-C₆ cycloalkyl, or each R⁹ is independently a C₁-C₆alkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆cycloalkyl, or C₃-C₈heterocycloalkyl groups of R⁹ are each optionally substituted with 1 to 3 substituents independently selected from —CN, R¹¹, —OR¹¹, —SR¹¹, —C(O)R¹¹, OC(O)R¹¹, —C(O)OR¹¹, —NR¹¹R¹², —C(O)NR¹¹R¹², —C(O)NR¹⁰H, —S(O)₂R¹¹, —S(O)R¹¹, —S(O)₂NR¹¹R¹², NR¹¹S(O)₂R¹¹, —P(O)(OR¹¹)₂ and —OP(O)(OR¹¹)₂;
   each R¹⁰ is independently selected from aryl, C₃-C₈cycloalkyl, C₃-C₈heterocycloalkyl and heteroaryl, wherein the aryl, C₃-C₈cycloalkyl, C₃-C₈heterocycloalkyl and heteroaryl groups are optionally substituted with 1 to 3 substituents selected from halogen, —R⁸, —OR⁸, -LR⁹, -LOR⁹, —N(R⁹)₂, —NR⁹C(O)R⁸, —NR⁹CO₂R⁸. —CO₂R⁸, —C(O)R⁸ and —C(O)N(R⁹)₂;
   R¹¹ and R¹² are independently selected from H, C₁-C₆alkyl, C₁-C₆heteroalkyl, C₁-C₆haloalkyl, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, aryl, heteroaryl, C₃-C₈cycloalkyl, and C₃-C₈heterocycloalkyl groups of R¹¹ and R¹² are each optionally substituted with 1 to 3 substituents independently selected from halogen, —CN, R⁸, —OR⁸, C(O)R⁸, OC(O)R⁸, —C(O)OR⁸, —N(R⁹)₂, —NRC(O)R⁸, —NRC(O)OR⁸, —C(O)N(R⁹)₂, C₃-C₈heterocycloalkyl, —S(O)₂R⁸, —S(O)₂N(R⁹)₂, —NR⁹S(O)₂R⁸, C₁-C₆haloalkyl and C₁-C₆haloalkoxy;
   or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
   ring A is an aryl or a heteroaryl, wherein the aryl and heteroaryl groups of Ring A are optionally substituted with 1 to 3 R^{A} groups, wherein each R^{A} is independently selected from —R⁸, —R⁷, —OR⁷, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂, —NR⁹C(S)N(R⁹)₂, —NR⁹CO₂R⁸, —NR⁹NR⁹C(O)R⁸, —NR⁹NR⁹C(O)N(R⁹)₂, —NR⁹NR⁹CO₂R⁸, —C(O)C(O)R⁸, —C(O)CH₂C(O)R⁸, —CO₂R⁸, —(CH₂)ₙCO₂R⁸, —C(O)R⁸, —C(S)R⁸, —C(O)N(R⁹)₂, —C(S)N(R⁹)₂, —OC(O)N(R⁹)₂, —OC(O)R⁸, —C(O)N(OR⁸)R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸, —CH═CHCO₂R⁸, —C(═NH)—N(R⁹)₂, and —(CH₂)ₙNHC(O)R⁸ or two adjacent R^{A} substituents on Ring A form a 5-6 membered ring that contains up to two heteroatoms as ring members;
   n is, independently at each occurrence, 0, 1, 2, 3, 4, 5, 6, 7 or 8;
   each m is independently selected from 1, 2, 3, 4, 5 and 6, and
   t is 1, 2, 3, 4, 5, 6, 7 or 8.

### Formulae (C), (D), (E), (G) and (H)

As discussed above, the TLR agonist can be of formula (C), (D), (E), (G) or (H).

The ‘parent’ compounds of formulae (C), (D), (E) and (H) are useful TLR7 agonists (see references 6-9 and 36-52) but are preferably modified herein by attachment of a phosphorus-containing moiety.

In some embodiments of formulae (C), (D) and (E) the compounds have structures according to formulae (C′), (D′) and (E′), shown below:

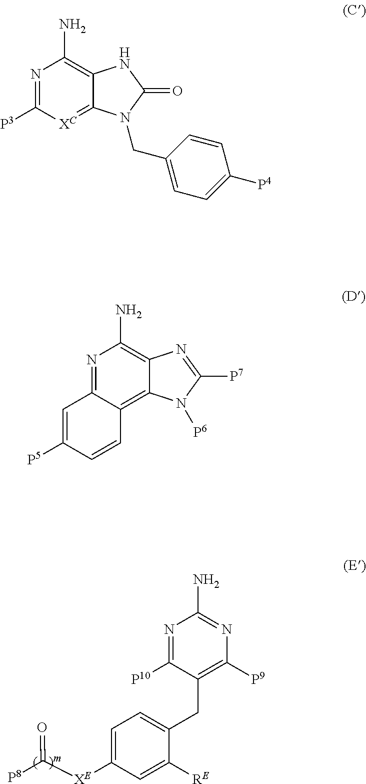

The embodiments of the invention of formulae (C), (D), (E) and (H) also apply to formulae (C′), (D′), (E′) and (H′).

In some embodiments of formulae (C), (D), (E), and (H): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (C): P³ is selected from C₁-C₆alkyl, CF₃, and —((CH₂)ₚO)_{q}(CH₂)ₚOₛ— and —Y-L-X—P(O)(OR^{X})(OR^{Y}); P⁴ is selected from —C₁-C₆alkylaryl and —Y-L-X—P(O)(OR^{X})(OR^{Y}); X^{C} is CH; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is 1 or 2.

In other embodiments of formulae (C), (D), (E), and (H): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (C): P³ is selected from C₁-C₆alkyl, CF₃, and —((CH₂)ₚO)_{q}(CH₂)ₚOₛ— and —Y-L-X—P(O)(OR^{X})(OR^{Y}); P⁴ is selected from —C₁-C₆alkylaryl and —Y-L-X—P(O)(OR^{X})(OR^{Y}); X^{C} is N; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

In other embodiments of formula (D): P⁵ is selected from C₁-C₆alkyl, and —Y-L-X—P(O)(OR^{X})(OR^{Y}).

In other embodiments of formula (D): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (D): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (E): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (E): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (E): X^{E} is CH₂, P⁸ is C₁-C₆alkoxy optionally substituted with —Y-L-X—P(O)(OR^{X})(OR^{Y}).

In other embodiments of formula (E): P⁹ is —NHC₁-C₆alkyl optionally substituted with OH and C₁-C₆alkyl, and —Y-L-X—P(O)(OR^{X})(OR^{Y}).

In some embodiments, a compound of formula (C) is not a compound in which P⁴ is —Y-L-X—P(O)(OR^{X})(OR^{Y}).

In some embodiments, in a compound of formula (C), P⁴ is selected from H, C₁-C₆alkyl, —C₁-C₆alkylaryl.

In some embodiments of formula (H): X^{H1}—X^{H2} is CR^{H2}R^{H3}, R^{H2} and R^{H3} are H, X^{H3} is N, X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In some embodiments of formula (H): X^{H1}—X^{H2} is CR^{H2}R^{H3}, R^{H2} and R^{H3} are H, X^{H3} is N, X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

The ‘parent’ compounds of formula (G) are useful TLR⁸ agonists (see references 10 & 11) but are preferably modified herein by attachment of a phosphorus-containing moiety to permit adsorption. In some embodiments of formula (G), the compounds have structures according to formula (G′);

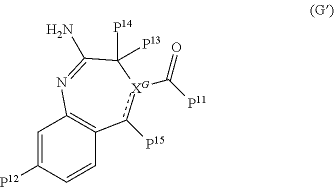

In some embodiments of formula (G) or (G′): X^{G} is C and [symbol] represents a double bond.

In some embodiments of formula (G) or (G′): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In some embodiments of formula (G) or (G′): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

### Pharmaceutical Compositions and Products

The invention provides various immunogenic compositions. These are ideally pharmaceutical compositions suitable for use in humans. Pharmaceutical compositions usually include components in addition to the TLR agonist, insoluble metal salt and/or immunogen e.g. they typically include one or more pharmaceutical carrier(s) and/or excipient(s). A thorough discussion of such components is available in reference 53.

Pharmaceutical compositions are preferably in aqueous form, particularly at the point of administration, but they can also be presented in non-aqueous liquid forms or in dried forms e.g. as gelatin capsules, or as lyophilisates, etc.

Pharmaceutical compositions may include one or more preservatives, such as thiomersal or 2-phenoxyethanol. Mercury-free compositions are preferred, and preservative-free vaccines can be prepared.

Pharmaceutical compositions can include a physiological salt, such as a sodium salt e.g. to control tonicity. Sodium chloride (NaCl) is typical, which may be present at between 1 and 20 mg/ml e.g. 10±2 mg/ml or 9 mg/ml. Other salts that may be present include potassium chloride, potassium dihydrogen phosphate, disodium phosphate dehydrate, magnesium chloride, calcium chloride, etc.

Pharmaceutical compositions can have an osmolality of between 200 mOsm/kg and 400 mOsm/kg, e.g. between 240-360 mOsm/kg, or between 290-310 mOsm/kg.

Pharmaceutical compositions may include compounds (with or without an insoluble metal salt) in plain water (e.g. w.f.i.) but will usually include one or more buffers. Typical buffers include: a phosphate buffer (except in the fifteenth aspect); a Tris buffer; a borate buffer; a succinate buffer; a histidine buffer (particularly with an aluminium hydroxide adjuvant); or a citrate buffer. Buffer salt s will typically be included in the 5-20 mM range. If a phosphate buffer is used then the concentration of phosphate ions should, in some embodiments, be <50 mM (see above) e.g. <10 mM.

Pharmaceutical compositions typically have a pH between 5.0 and 9.5 e.g. between 6.0 and 8.0.

Pharmaceutical compositions are preferably sterile.

Pharmaceutical compositions preferably non-pyrogenic e.g. containing <1 EU (endotoxin unit, a standard measure) per dose, and preferably <0.1 EU per dose.

Pharmaceutical compositions are preferably gluten free.

Pharmaceutical compositions are suitable for administration to animal (and, in particular, human) patients, and thus include both human and veterinary uses. They may be used in a method of raising an immune response in a patient, comprising the step of administering the composition to the patient. Compositions may be administered before a subject is exposed to a pathogen and/or after a subject is exposed to a pathogen.

Pharmaceutical compositions may be prepared in unit dose form. In some embodiments a unit dose may have a volume of between 0.1-1.0 ml e.g. about 0.5 ml.

The invention also provides a delivery device (e.g. syringe, nebuliser, sprayer, inhaler, dermal patch, etc.) containing a pharmaceutical composition of the invention e.g. containing a unit dose. This device can be used to administer the composition to a vertebrate subject.

The invention also provides a sterile container (e.g. a vial) containing a pharmaceutical composition of the invention e.g. containing a unit dose.

The invention also provides a unit dose of a pharmaceutical composition of the invention.

The invention also provides a hermetically sealed container containing a pharmaceutical composition of the invention. Suitable containers include e.g. a vial.

The invention also provides a kit comprising first and second kit components, wherein: (i) the first kit component comprises an insoluble metal salt and at least one S. aureus antigen; and (ii) the second kit component comprises a TLR agonist. The second component ideally does not include an insoluble metal salt and/or does not include a S. aureus antigen. The first and second components can be combined to provide a composition suitable for administration to a subject.

The invention also provides a kit comprising first and second kit components, wherein: (i) the first kit component comprises an insoluble metal salt and a TLR agonist; and (ii) the second kit component comprises at least one S. aureus antigen. The second component ideally does not include an insoluble metal salt and/or a TLR agonist. In some embodiments, the second component is lyophilised. The first and second components can be combined to provide a pharmaceutical composition suitable for administration to a subject.

The invention also provides a kit comprising first and second kit components, wherein: (i) the first kit component comprises at least one S. aureus antigen and a TLR agonist; and (ii) the second kit component comprises an insoluble metal salt. The second component ideally does not include a S. aureus antigen and/or a TLR agonist. The first and second components can be combined to provide a pharmaceutical composition suitable for administration to a subject.

In some embodiments these kits comprise two vials. In other embodiments they comprise one ready-filled syringe and one vial, with the contents of the syringe being mixed with the contents of the vial prior to injection. A syringe/vial arrangement is useful where the vial's contents are lyophilised. Usually, though, the first and second kit components will both be in aqueous liquid form.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. by an inhaler, using a fine powder or a spray. The composition may be prepared as a suppository or pessary. The composition may be prepared for nasal, aural or ocular administration e.g. as a spray or drops. The composition may be in kit form, designed such that a combined composition is reconstituted just prior to administration to a patient. Such kits may comprise one or more antigens in liquid form and one or more lyophilised antigens. Injectables for intramuscular administration are typical.

Compositions comprise an effective amount of a TLR agonist i.e. an amount which, when administered to an individual, either in a single dose or as part of a series, is effective for enhancing the immune response to a co-administered S. aureus antigen. This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. The amount will fall in a relatively broad range that can be determined through routine trials. An amount of between 1-1000 μg/dose can be used e.g. from 5-100 μg per dose or from 10-100 μg per dose, and ideally ≦300 μg per dose e.g. about 5 μg, 10 μg, 20 μg, 25 μg, 50 μg or 100 μg per dose. Thus the concentration of a TLR agonist in a composition of the invention may be from 2-2000 μg/ml e.g. from 10-200 μg/ml, or about 10, 20, 40, 50, 100 or 200 μg/ml, and ideally ≦600 μg/ml.

### Methods of Treatment, and Administration of Immunogenic Compositions

The invention provides a method of raising an immune response in a subject, comprising the step of administering to the subject a composition of the invention.

The invention also provides a composition of the invention, for use in a method of raising an immune response in a subject.

The invention also provides the use of a TLR agonist, insoluble metal salt and S. aureus antigen(s) in the manufacture of a medicament for raising an immune response in a subject.

The invention also provides the use of (i) a TLR agonist as defined herein and (ii) an insoluble metal salt and (iii) one or more S. aureus antigens, in the manufacture of a medicament (e.g. a vaccine) for raising an immune response in a subject.

The invention is suitable for raising immune responses in human or non-human animal (in particular mammal) subjects. Compositions prepared according to the invention may be used to treat both children and adults.

The immune response stimulated by these methods and uses will generally include an antibody response, preferably a protective antibody response. The immune response can also include a cellular response. Methods for assessing antibody and cellular immune responses after immunisation are well known in the art.

Treatment can be by a single dose schedule or a multiple dose schedule. Multiple doses may be used in a primary immunisation schedule and/or in a booster immunisation schedule. Administration of more than one dose (typically two doses) is particularly useful in immunologically naïve patients. Multiple doses will typically be administered at least 1 week apart (e.g. about 2 weeks, about 3 weeks, about 4 weeks, about 6 weeks, about 8 weeks, about 10 weeks, about 12 weeks, etc.).

### Chemical Groups

Unless specifically defined elsewhere, the chemical groups discussed herein have the following meaning when used in present specification:

The term “alkyl” includes saturated hydrocarbon residues including:
linear groups up to 10 atoms (C₁-C₁₀), or of up to 6 atoms (C₁-C₆), or of up to 4 atoms (C₁-C₄).
Examples of such alkyl groups include, but are not limited, to C₁-methyl, C₂-ethyl, C₃-propyl and C₄-n-butyl.
branched groups of between 3 and 10 atoms (C₃-C₁₀), or of up to 7 atoms (C₃-C₇), or of up to 4 atoms (C₃-C₄). Examples of such alkyl groups include, but are not limited to, C₃— iso-propyl, C₄-sec-butyl, C₄-iso-butyl, C₄-tert-butyl and C₅-neo-pentyl.

The term “alkylene” refers to the divalent hydrocarbon radical derived from an alkyl group, and shall be construed in accordance with the definition above.

The term “alkenyl” includes monounsaturated hydrocarbon residues including:
linear groups of between 2 and 6 atoms (C₂-C₆). Examples of such alkenyl groups include, but are not limited to, C₂-vinyl, C₃-1-propenyl, C₃-allyl, C₄-2-butenyl
branched groups of between 3 and 8 atoms (C₃-C₈). Examples of such alkenyl groups include, but are not limited to, C₄-2-methyl-2-propenyl and C₆-2,3-dimethyl-2-butenyl.

The term alkenylene refers to the divalent hydrocarbon radical derived from an alkenyl group, and shall be construed in accordance with the definition above.

The term “alkoxy” includes O-linked hydrocarbon residues including:
linear groups of between 1 and 6 atoms (C₁-C₆), or of between 1 and 4 atoms (C₁-C₄). Examples of such alkoxy groups include, but are not limited to, C₁-methoxy, C₂-ethoxy, C₃-n-propoxy and C₄-n-butoxy.
branched groups of between 3 and 6 atoms (C₃-C₆) or of between 3 and 4 atoms (C₃-C₄). Examples of such alkoxy groups include, but are not limited to, C₃-iso-propoxy, and C₄-sec-butoxy and tert-butoxy.

Halo is selected from Cl, F, Br and I. Halo is preferably F.

The term “aryl” includes a single or fused aromatic ring system containing 6 or 10 carbon atoms; wherein, unless otherwise stated, each occurrence of aryl may be optionally substituted with up to 5 substituents independently selected from (C₁-C₆)alkyl, (C₁-C₆)alkoxy, OH, halo, CN, COOR¹⁴, CF₃ and NR¹⁴R¹⁵; as defined above. Typically, aryl will be optionally substituted with 1, 2 or 3 substituents. Optional substituents are selected from those stated above. Examples of suitable aryl groups include phenyl and naphthyl (each optionally substituted as stated above). Arylene refers the divalent radical derived from an aryl group, and shall be construed in accordance with the definition above.

The term “heteroaryl” includes a 5, 6, 9 or 10 membered mono- or bi-cyclic aromatic ring, containing 1 or 2 N atoms and, optionally, an NR¹⁴ atom, or one NR¹⁴ atom and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, COOR¹⁴, CF₃ and NR¹⁴R¹⁵; as defined below. Examples of suitable heteroaryl groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above). Heteroarylene refers the divalent radical derived from heteroaryl, and shall be construed in accordance with the definition above.

The term “heterocyclyl” is a C-linked or N-linked 3 to 10 membered non-aromatic, mono- or bi-cyclic ring, wherein said heterocycloalkyl ring contains, where possible, 1, 2 or 3 heteroatoms independently selected from N, NR¹⁴, S(O)_{q} and O; and said heterocycloalkyl ring optionally contains, where possible, 1 or 2 double bonds, and is optionally substituted on carbon with 1 or 2 substituents independently selected from (C₁-C₆)alkyl, (C₁-C₆)alkoxy, OH, CN, CF₃, halo, COOR¹⁴, NR¹⁴R¹⁵ and aryl.

In the above definitions R¹⁴ and R¹⁵ are independently selected from H and (C₁-C₆)alkyl.

When a structural formula is defined with a substituent attached to the core of the molecule by an unspecified, or “floating” bond, for example, as for the group P³ in the case of formula (C), this definition encompasses the cases where the unspecified substituent is attached to any of the atoms on the ring in which the floating bond is located, whilst complying with the allowable valence for that atom.

In the case of compounds of the invention which may exist in tautomeric forms (i.e. in keto or enol forms), for example the compounds of formula (C) or (H), reference to a particular compound optionally includes all such tautomeric forms.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Phosphorous-containing groups employed with the invention may exist in a number of protonated and deprotonated forms depending on the pH of the surrounding environment, for example the pH of the solvent in which they are dissolved. Therefore, although a particular form may be illustrated it is intended, unless otherwise mentioned, for these illustrations to merely be representative and not limiting to a specific protonated or deprotonated form. For example, in the case of a phosphate group, this has been illustrated as —OP(O)(OH)₂ but the definition includes the protonated forms —[OP(O)(OH₂)(OH)]⁺ and —[OP(O)(OH₂)₂]²⁺ that may exist in acidic conditions and the deprotonated forms —[OP(O)(OH)(O)]⁻ and [OP(O)(O)₂]²⁻ that may exist in basic conditions.

Compounds disclosed herein can exist as pharmaceutically acceptable salts. Thus, the compounds may be used in the form of their pharmaceutically acceptable salts i.e. physiologically or toxicologically tolerable salt (which includes, when appropriate, pharmaceutically acceptable base addition salts and pharmaceutically acceptable acid addition salts).

### BRIEF DESCRIPTION OF THE DRAWINGS

FIG. 1 shows IgG titers, after 3 intramuscular injections, against (A) Hla-H35L (B) EsxAB (C) Sta006 (D) Sta011. In each panel the four groups are, from left to right: Al—H adjuvant alone; Al—H/K2 alone; Combo-1+Al—H; Combo-1+Al—H/K2. The ** indicates a statistically significant difference (p<0.05) against Combo-1+Al—H.

FIG. 2 shows (A) interferon-γ and (B) IL-4/IL-13 responses in immunised mice. Groups A to I on the X-axis received: (A) saline; (B) Al—H alone; (C) Al—H/K2 alone; (D) unadjuvanted antigens; (E) antigens adjuvanted with Al—H; (F) antigens adjuvanted with Al—H/K2 at 1 μg K2; (G) as (F) but with 5 μg K2; (H) as (F) but with 25 μg K2; (I) as (F) but with 50 μg K2.

FIG. 3 shows CFU (log) in kidneys in an abscess model.

FIGS. 4 to 7 show antibody titers in Balb/C mice. The 10 groups, from left to right, are: four negative controls (saline and/or buffers alone); unadjuvanted Combo-1 antigens (after 2 doses, and after 3 doses); Combo-1 with Al—H adjuvant (2 & 3 doses); and Combo-1 with Al—H/K2 (2 & 3 doses). FIG. 4 shows anti-HLA responses; FIG. 5 shows anti-EsxAB responses; FIG. 6 shows anti-Sta006 responses; and FIG. 7 shows anti-Sta011 responses. Stars indicate statistical significance by the Mann-Whitney test (‘**’, p<0.01).

FIGS. 8 to 12 show % survival in mice after immunisation with Combo-1 with various adjuvants.

FIG. 13 shows areas (mm²) of abscesses (13A & 13C) or dermonecrosis (13B & 13D) in mice immunised with Combo-1 adjuvanted with Al—H (13A & 13B0 or Al—H/K2 (13C & 13D). In each group the squares show data for Combo-1, whereas circles show data for adjuvant alone. A * indicates a statistically significant difference between mice receiving adjuvant or antigen+adjuvant. The x-axis shows days post-infection with strain USA300.

FIG. 14 shows survival rates (%) in mice challenged with (A) Newman (B) MW2 or (C) LAC. In each case the left-hand column of a pair is a negative control without antigen, and the right-hand column is for Combo-1 with (i) no adjuvant (ii) Al—H (iii) MF59 or (iv) Al—H/K2.

FIG. 15 shows SDS-PAGE analysis of Cys(+) and Cys(−) formulations with Al—H/K2. Lane 1 has molecular weight markers. Lanes 2-5 are Cys(+) antigens Hla-H35L, EsxAB, Sta006 and Sta011 (in order); lanes 8-11 are the Cys(−) antigens. Lane 6 shows desorbed Cys(+) antigens, and lane 12 shows the same for Cys(−). Lanes 7 and 13 show TCA-treated supernatants.

### MODES FOR CARRYING OUT THE INVENTION

### Vaccine Preparation and Administration

References 35 and 54 disclose TLR7 agonists having formula (K) as discussed above. One of these compounds, 3-(5-amino-2-(2-methyl-4-(2-(2-(2-phosphonoethoxy)ethoxy)ethoxy)phenethyl)benzo [f]-[1,7]naphthyridin-8-yl)propanoic acid is referred to hereafter as compound “K2”:

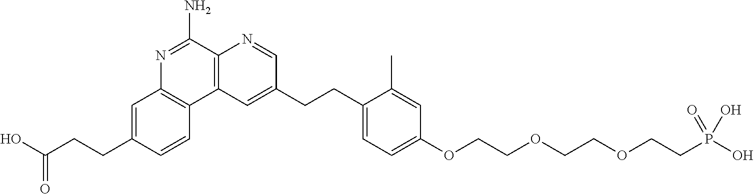 [SMILES: Cc1cc(OCCOCCOCCP(=O)(O)O)ccc1CCc1cnc2c(N)nc3cc(CCC(=O)O)ccc3c2c1]

Compound K2 is added to water at 4 mg/ml, then 1M NaOH is added to ensure full solubilisation, with stirring for 15 minutes at room temperature. This material is added to a suspension of aluminium hydroxide adjuvant (Al—H; 2 mg/ml) to give the desired final concentration. This mixture is shaken for 2 hours at ambient temperature to ensure full adsorption, and then histidine buffer components are added (10 mM histidine buffer, pH 6.5).

The compound can also be used as an arginine salt monohydrate (obtained by mixing 98 mg of the compound with 1.7 ml of 0.1M arginine in 80/20 methanol/water to give a 57 mg/mL solution, followed by addition of 7 ml ethanol to precipitate the salt) in which case it is seen that the NaOH is not required for solubilisation prior to mixing with the Al—H.

Four different mixtures are prepared, giving a final K2 concentration of 10, 50, 250 or 500 μg/ml (to provide a 1, 5, 25 or 50 μg dose of K2 in a 1001 dosage volume); the Al—H concentration is always 2 mg/ml. At all strengths >95% of compound K2 is adsorbed to the Al—H. The adsorbed adjuvant is referred to hereafter as “Al—H/K2”.

The “Combo-1” vaccine from reference 1 includes a mixture of four polypeptides (EsxAB, Sta006, Sta011, and Hla-H35L) having amino acid sequences SEQ ID NOs: 7, 8, 27 and 32. These four polypeptides are mixed sequentially with Al—H/K2 to give a final dose of 1 μg or 10 μg of each polypeptide (10 μg/mL or 100 μg/mL). The order in which the polypeptides is added has little effect. In the resulting mixtures the K2 compound and the four polypeptides are all stably adsorbed to the aluminium hydroxide adjuvant, and the degree of adsorption (>80% in all cases) is essentially the same with Al—H/K2 as with Al—H alone. Osmolality for all compositions was between 260-285 mOsm/kg, and pH was between 6.6-6.9 (pH and osmolality are slightly higher for the 10 μg polypeptide mixtures). Compound K2 remains >95% adsorbed in the presence of the adsorbed polypeptides.

With the four adjuvant strengths and two antigen strengths, 8 different formulations were prepared.

Female Balb/c mice (16 per group) were immunized intramuscularly 3 times with the same formulation, at days 0, 14 and 28. Sera were taken prior to the each immunisation, and again on day 39, for analysis of antigen-specific antibody titers. On day 40 four mice per group were sacrificed for analysis of antigen-specific T-cell responses (spleen cells were stimulated with the four antigens, singly or in combination, and cytokine production was measured on CD4+ and CD8+ T cells; antigen-specific T-cell proliferation was evaluated by Click-iT EdU assay). The remaining 12 mice in each group were challenged with 2−3×10⁸ CFU of Newman strain S. aureus, administered in 100 μg interperitoneally. The efficacy of the vaccine in protecting mice against challenge in this sepsis model was assessed as the percentage of surviving mice 2 weeks later (day 54).

### Results

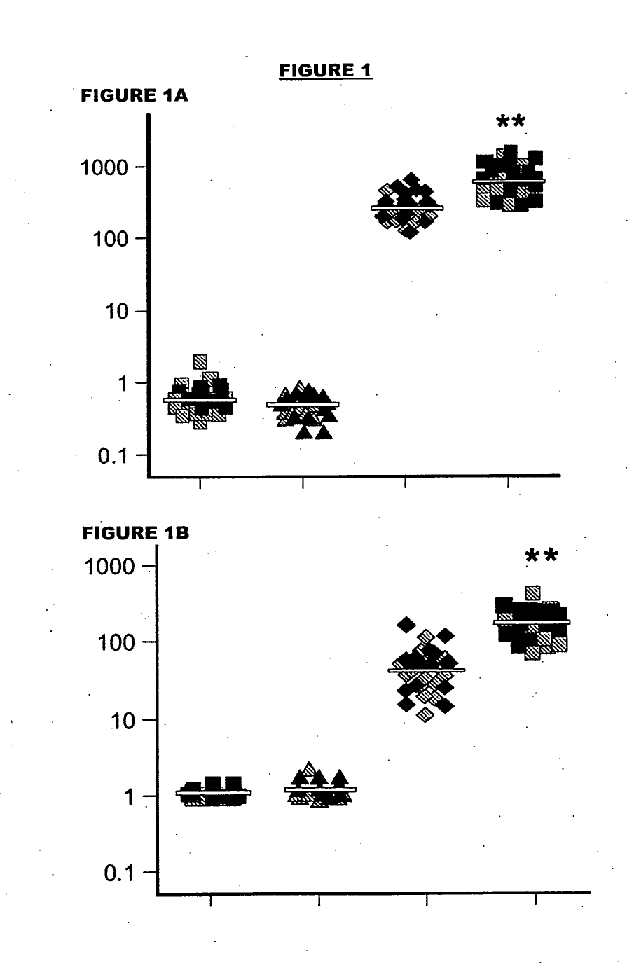

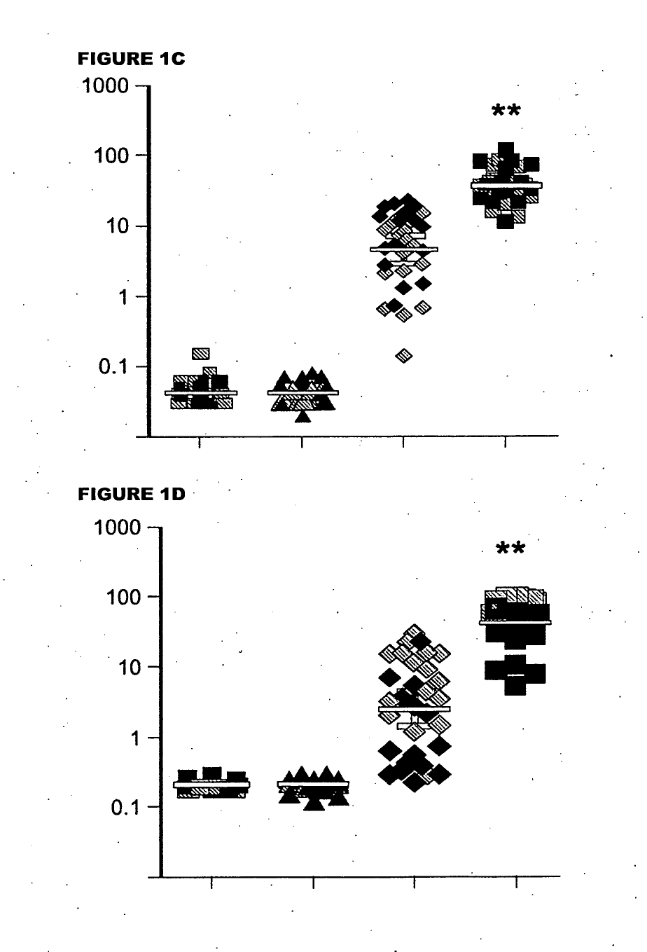

FIG. 1 shows IgG titers against the individual polypeptides at day 39 after 3× administration of the polypeptides at 10 μg each with Al—H/K2 (25 μg of K2). For all four polypeptides the titer obtained using Al—H/K2 was higher than the titer obtained using Al—H alone (**, p<0.05).

The compositions with 10×-less antigen gave comparable results but with lower antibody titers and weaker T-cell responses. Similar results were seen using 1, 5, or 50 μg of K2.

In relation to recall-specific T cell responses, the use of Al—H/K2 gave more antigen-specific CD4⁺ T cells that produce TNF-α, IL-2 and IFN-γ compared to immunisation with unadjuvanted antigens or with antigens adjuvanted with Al—H alone. FIG. 2 shows interferon-γ and IL-4/IL-13 responses. The percentage of antigen-specific CD4⁺ T cells that produce IL-4 and IL-13 was higher (although not statistically significant) when using Al—H compared to unadjuvanted Combo-1, but immunization using the Al—H/K2 combination reduced this effect at all doses except the lowest, indicating that the Th2-polarizing effect of Al—H was counterbalanced by the Th1-polarizing effect of the TLR7 agonist.

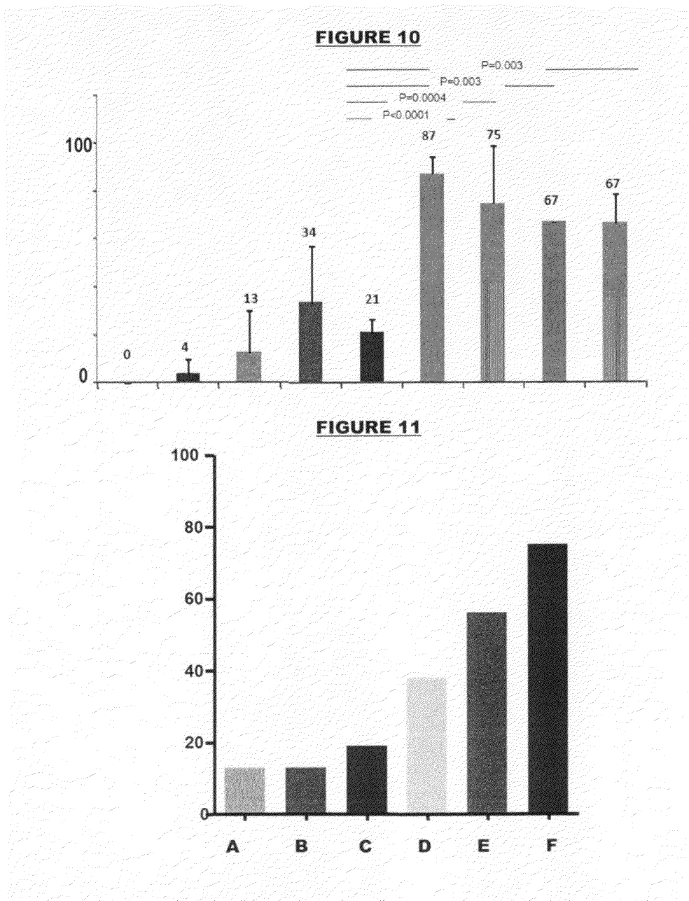

Protection data from two pooled experiments with Combo-1 (10 μg of each polypeptide) in a sepsis model with Balb-c mice, using intramuscular immunisation, were as follows, showing the proportion of animals surviving after 15 days (FIG. 10), and the median survival length (days):

| | | | Survival | P value |
|---|---|---|---|---|
| Antigen | Adjuvant | % survival | days | (Fisher) |
| − | — | 0 | 1 | — |
| − | Al—H | 4 | 1 | — |
| − | Al—H + 50 μg K2 | 13 | 1 | — |
| + | — | 34 | 7 | — |
| + | Al—H | 21 | 7.5 | — |
| + | Al—H + 50 μg K2 | 87 | 15 | <0.0001 |
| + | Al—H + 25 μg K2 | 75 | 15 | 0.0004 |
| + | Al—H + 5 μg K2 | 67 | 15 | 0.003 |
| + | Al—H + 1 μg K2 | 67 | 15 | 0.003 |

Thus survival was better than Al—H alone when using the Al—H/K2 combination. In all cases the addition of K2 (1-50 μg) improved survival from 21% to 67-87%, with statistical significance (p=0.003 or better)

In summary, the Al—H/K2 adjuvant combination increased IgG titers to all four antigens, increased the frequency of cytokine-producing CD4 T cells; balanced the Th2 bias of Al—H alone (higher IFNγ, lower IL-4/IL-13), and increased survival compared to adjuvanting with Al—H alone.

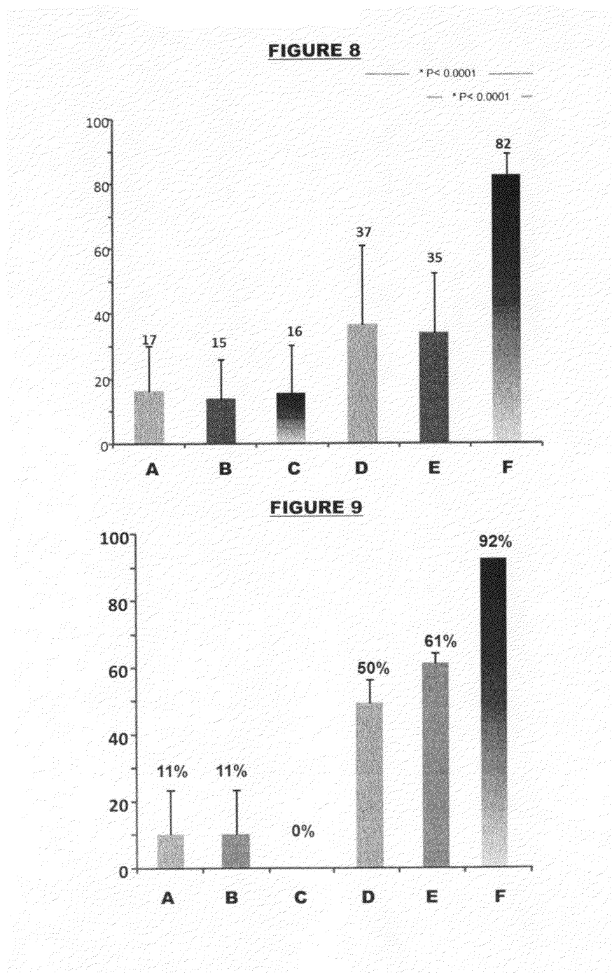

In similar experiments with Balb/C mice (>32 per group) using Combo-1 (10 μg of each antigen) and 50 μg of K2, with lethal challenge by Newman strain, survival was as follows (FIG. 8):

| Group | Antigen | Adjuvant | % survival |
|---|---|---|---|
| A | − | — | 17 |
| B | − | Al—H | 15 |
| C | − | Al—H + K2 | 16 |
| D | + | — | 37 |
| E | + | Al—H | 35 |
| F | + | Al—H + K2 | 82 |

The survival rate in the Al—H/K2 group was statistically superior to all other groups, p<0.0001.

In a sepsis model with CD1 mice (12-44 per group), using intramuscular immunisation with Combo-1 (10 μg of each antigen) and 50 μg of K2, with challenge by Newman strain, survival after 15 days was as follows (see also FIG. 9—groups A to H from left to right):

| | | | | | Survival |
|---|---|---|---|---|---|
| Group | Antigen | Adjuvant | % survival | % P.E. † | days |
| A | − | — | 11 | — | 1 |
| B | − | Al—H | 11 | — | 1 |
| C | − | Al—H + K2 | 0 | — | 1 |
| D | + | — | 50 | 44 ** | 14.5 ** |
| E | + | Al—H | 61 | 56 ** | 15 ** |
| F | + | Al—H + K2 | 92 | 92 ** | 15 ** |

† P.E. = protective efficacy = 1 − (% dead vaccinated/% dead control)
** P value (Fisher for % P.E.; Mann-Whitney for days) < 0.0001
* P value (Fisher) 0.0006

Results using USA300 (LAC) as the challenge strain were as follows (FIG. 11):

| | | | | Survival | |
|---|---|---|---|---|---|
| Group | % survival | % P.E. | P value | days | P value |
| A | 13 | — | — | 1 | — |
| B | 13 | — | — | 1 | — |
| C | 19 | — | — | 1 | — |
| D | 38 | 29 | 0.11 | 2 | 0.0046 |
| E | 56 | 49 | 0.011 | 15 | 0.0011 |
| F | 78 | 69 | 0.0019 | 15 | 0.0002 |

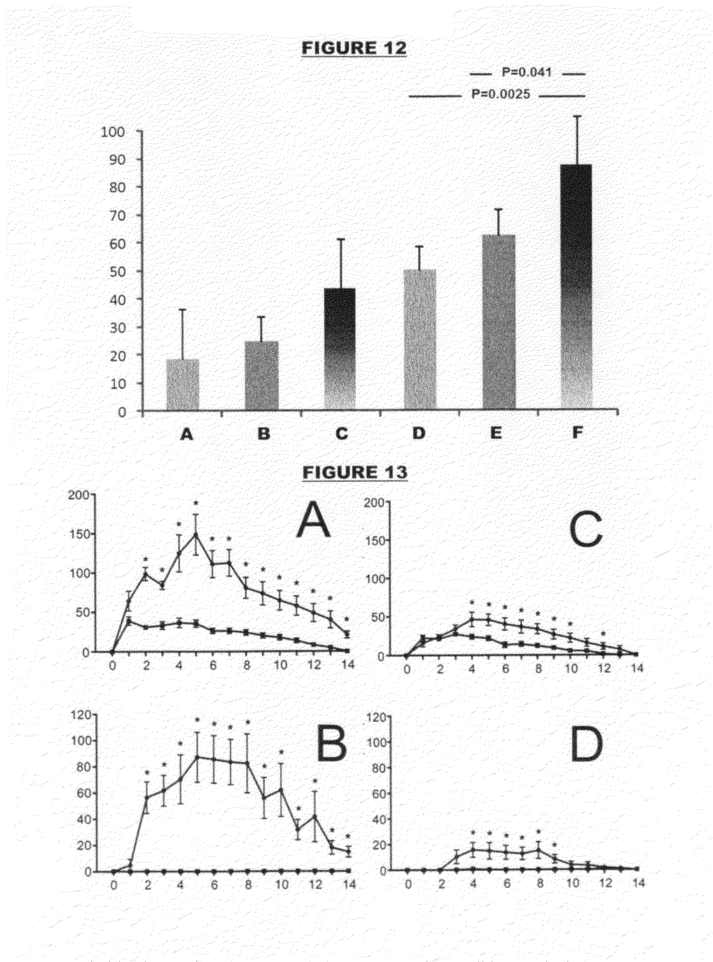

Results using USA400 (MW2) as the challenge strain were as follows (FIG. 12):

| | | | | Survival | |
|---|---|---|---|---|---|
| | | | P value | days | P value |
| Group | % survival | % P.E. | (Fisher) | (median) | (Mann-Whitney) |
| A | 19 | — | — | 1 | — |
| B | 25 | — | — | 1 | — |
| C | 44 | — | — | 2.5 | — |
| D | 50 | 38 | 0.0084 | 10.5 | <0.0001 |
| E | 63 | 51 | 0.0017 | 15 | 0.0001 |
| F | 88 | 79 | 0.0002 | 15 | <0.0001 |

Overall, therefore, survival rates were as follows for the three challenge strains:

| Group | Antigen | Adjuvant | Newman | USA300 | USA400 |
|---|---|---|---|---|---|
| A | − | — | 11 | 13 | 19 |
| B | − | Al—H | 11 | 13 | 25 |
| C | − | Al—H + K2 | 0 | 19 | 44 |
| D | + | — | 50 | 38 | 50 |
| E | + | Al—H | 61 | 56 | 63 |
| F | + | Al—H + K2 | 92 | 78 | 88 |

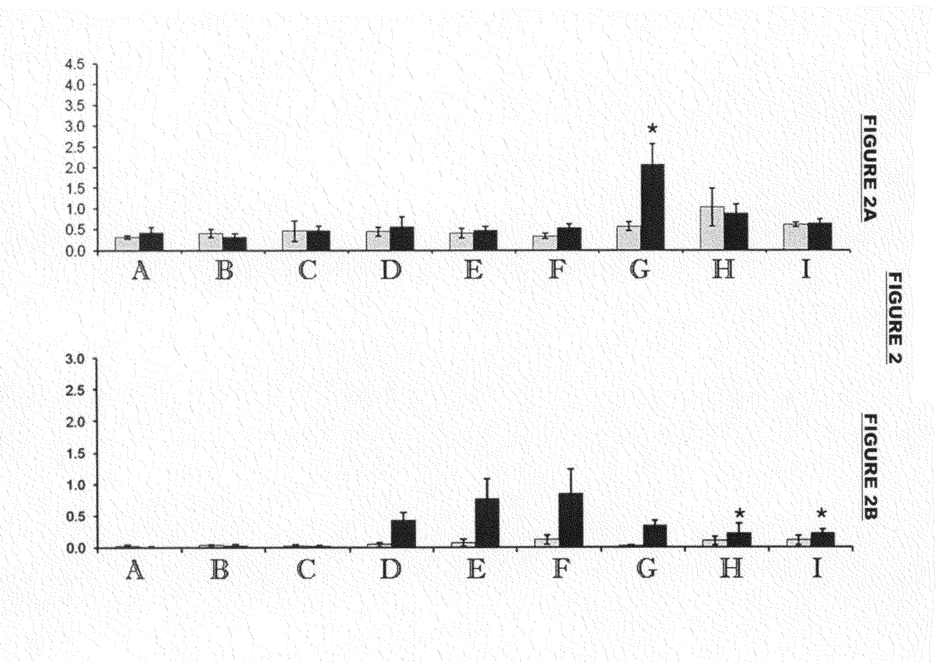

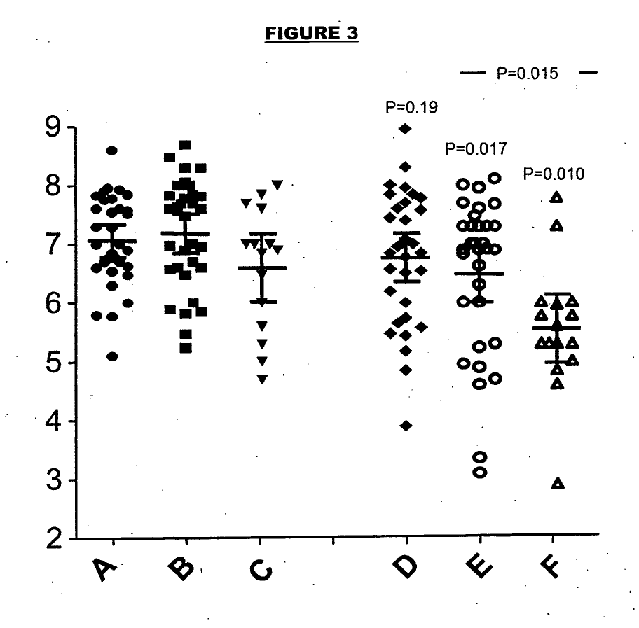

FIG. 3 shows results from an abscess model using CD1 mice with the same six treatments (A to F). The best results were seen in group F.

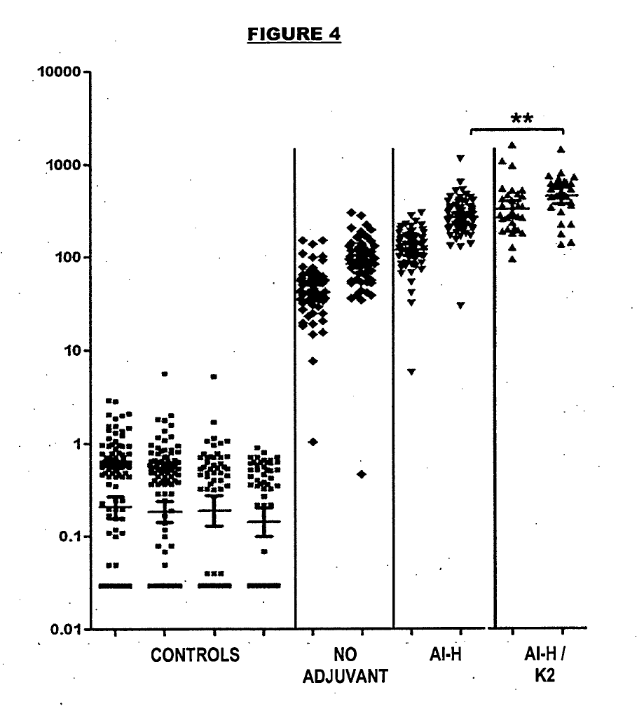

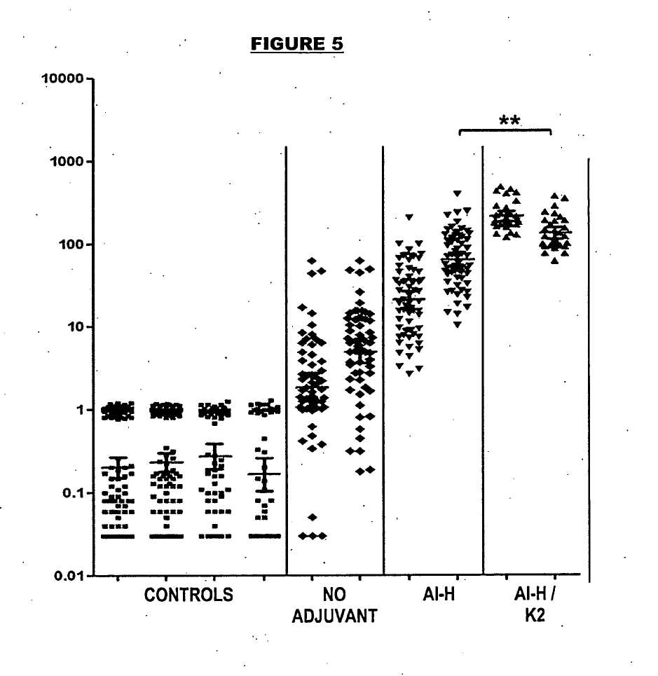

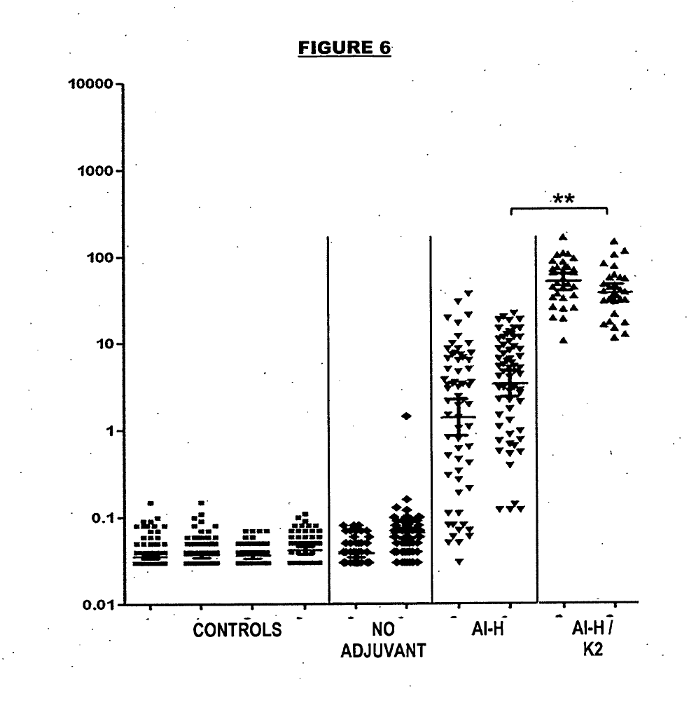

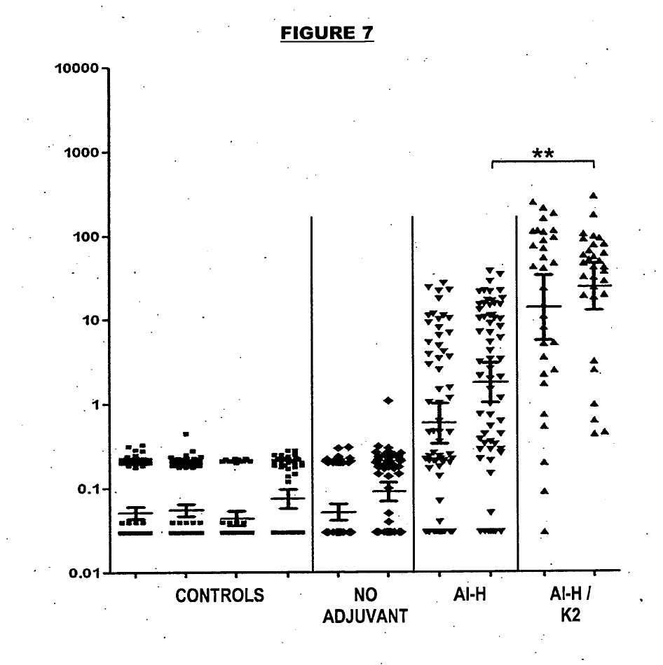

FIGS. 4 to 7 shows antibody titers in Balb/C mice against each of the four separate antigens in Combo-1. In all cases the addition of K2 improved responses compared with Al—H alone.

The magnitude and kinetics of the immune response were improved when using the Al—H/K2 combination. For all four antigens in “Combo-1” final titres were higher with this combination than with Al—H alone. Moreover, peak titres were reached after 2 immunizations with Al—H/K2, whereas the other tested adjuvants required 3 doses to reach the peak. These results were seen in both Balb/C and CD1 mice.

The improved kinetics were also seen when measuring protection in the sepsis model. Mice who received Combo-1 with the Al—H/K2 adjuvant were >95% protected after a single immunisation.

The Al—H/K2 mixture also changed the balance of T cells elicited by the vaccine. Whereas Al—H alone induced a mixed Th1/Th2 CD4⁺ T cell response, the addition of K2 shifted the response to a mixed Th1/Th17 response, including an IFN-γ response. Furthermore, compared to an unadjuvanted vaccine the use of Al—H alone did not increase cytokine and proliferation responses, whereas both of these responses were increased by the use of Al—H/K2.

FIG. 13 shows the development of abscesses after infection with strain USA300 in a skin infection model. The mice were immunised intramuscularly with the Combo-1 mixture (10 μg of each antigen) with Al—H with or without K2 (50 μg). As shown in FIG. 13, with both Al—H (FIGS. 13A & 13B) and Al—H/K2 (13C & 13D), Combo-1 (▪) significantly reduces abscess area (13A & 13C) and dermonecrosis area (13B & 13D) relative to controls (). Abscesses were smaller in the mice who were immunised with Combo-1 plus Al—H/K2 (FIG. 13B) than with Al—H (FIG. 13A).

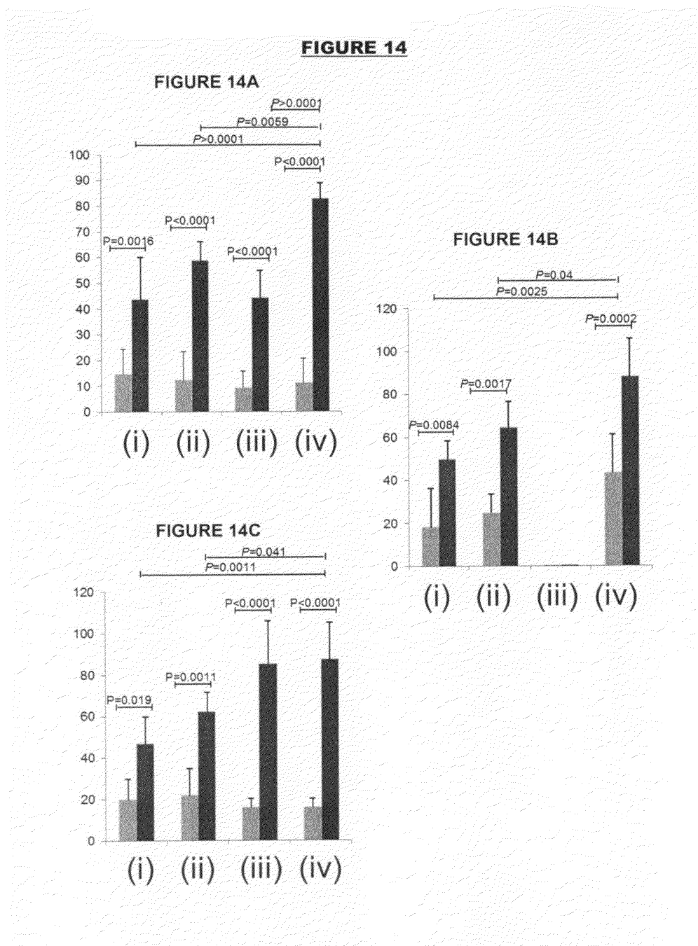

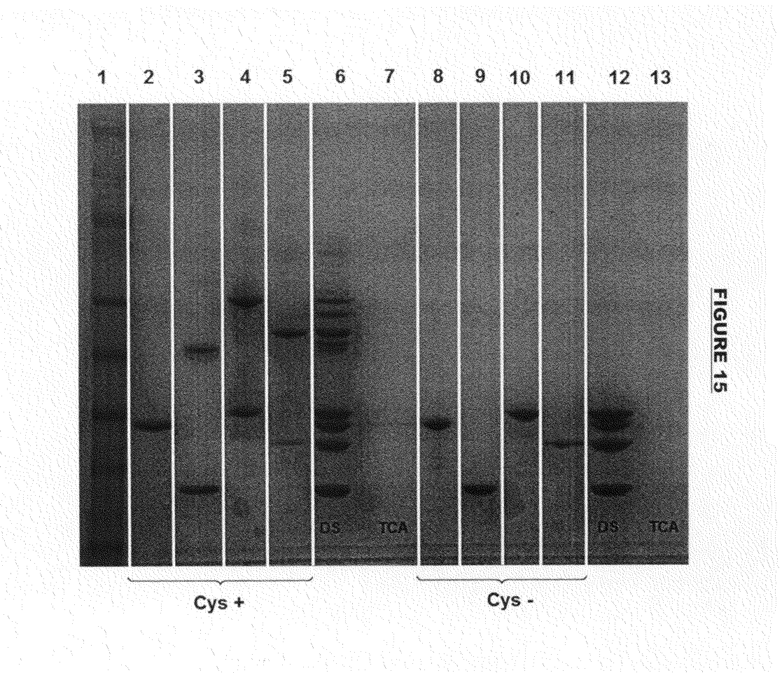

FIG. 14 shows survival data of CD1 mice immunised at days 0 & 14 with Combo-1 with (i) no adjuvant (ii) Al—H (iii) MF59 or (iv) Al—H/K2. These mice were challenged intraperitoneally at day 24 with (A) Newman (B) MW2 or (C) LAC strain. For each strain the highest survival rate was seen when using Al—H/K2 (more than 80% in each case), and for each strain the addition of K2 to Al—H provided a statistically significant improvement in survival rates.

Thus the use of Al—H/K2 significantly improves the behaviour of Combo-1 relative to Al—H alone.

### Cysteine Removal

The amino acid sequences of the Sta006, Sta011 and EsxAB antigens in the “Combo-1” vaccine were modified to remove their cysteine residues, to avoid formation of homodimers and heterodimers and thereby improve consistency of antigen formulations. Thus SEQ ID NOs: 7, 8, and 32 were converted to SEQ ID NOs: 44, 45 and 46. These Cys-free polypeptides were combined with HlaH35L (SEQ ID NO: 27) to make a “Cys(−)” version of “Combo-1”. Immunogenicity of the Cys(−) Combo-1 formulation was assessed in CD1 mice using Al—H/K2. The adjuvanted Cys(−) combination was immunogenic and elicited good antibody and T-cell responses in the mice.

Adsorption of the Cys(+) and Cys(−) combinations to Al—H/K2 was compared. 2 mg/ml Al—H and 0.5 mg/ml K2 in 10 mM histidine buffer (pH 6.5) were combined, then the antigens were added at 100 μg/ml each and left for 15 minutes to adsorb at room temperature. The two antigen formulations were assessed for adsorption after storage overnight at 4° C., and also treated to desorb the antigens for comparison.

SDS-PAGE was used to evaluate antigen adsorption. FIG. 15 shows free Cys(+) antigens in lanes 2-5 and free Cys(−) antigens in lanes 8-11. High-MW dimers are visible with the Cys(+) antigens, but are absent from the Cys(−) antigens. Lanes 7 and 13 show TCA-treated supernatants after centrifugation, and the absence of visible bands confirms that the proteins are fully adsorbed. Lanes 6 and 12 show the formulations after treatment with desorption buffer, confirming that the antigens can be extracted intact, without degradation.

It will be understood that the invention has been described by way of example only and modifications may be made whilst remaining within the scope and spirit of the invention.

### REFERENCES

[1] WO2010/119343.
[2] WO2012/031140.
[3] Burrell et al. (1999) Vaccine 17:2599-603.
[4] Rosenberg et al. (2010) J Immunol 184:136.20.
[5]U.S. Pat. No. 4,666,886.
[6] WO2009/118296.
[7] WO2008/005555.
[8] WO2009/111337.
[9] WO2009/067081.
[10] WO2007/040840.
[11] WO2010/014913.
[12] UK patent application GB-A-2220211.
[13] Myers et al. (1990) pages 145-156 of Cellular and molecular aspects of endotoxin reactions. [14] Ulrich (2000) Chapter 16 (pages 273-282) of reference 19.
[15] Johnson et al. (1999) J Med Chem 42:4640-9.
[16] Baldrick et al. (2002) Regulatory Toxicol Pharmacol 35:398-413.
[17] WO 94/21292.
[18]Vaccine Design . . . (1995) eds. Powell & Newman. ISBN: 030644867X. Plenum. [19] Vaccine Adjuvants: Preparation Methods and Research Protocols (Volume 42 of Methods in Molecular Medicine series). ISBN: 1-59259-083-7. Ed. O'Hagan.
[20] Clausi et al. (2008) J Pharm Sci DOI 10.1002/jps.21390.
[21] Sebulsky & Heinrichs (2001) J Bacteriol 183:4994-5000.
[22] Sebulsky et al. (2003) J Biol Chem 278:49890-900.
[23] Rable & Wardenburg (2009) Infect Immun 77:2712-8.
[24] WO2007/145689.
[25] WO2009/029831.
[26] WO2011/015590.
[27] WO2011/007004.
[28] Kim et al. (2010) Vaccine doi:10.1016/j.vaccine.2010.02.097
[29] WO2005/079315.
[30] WO2008/152447.
[31] WO2005/009379.
[32] WO2005/009378.
[33] WO2011/015591
[34] WO2011/119759.
[35] WO2011/027222.
[36] WO2007/034917.
[37] WO2007/034173.
[38] WO2008/114817.
[39] US2009-0105212.
[40] US2009-0118263.
[41] US2009-0143400.
[42] US2009-0192153.
[43] WO2007/093901.
[44] WO2009/019553.
[45] US2009/0221631.
[46] WO2008/004948.
[47] WO2008/135791.
[48] US2009/0099216.
[49] US2009/0202484.
[50] WO2008/101867.
[51] WO2010/077613.
[52] US2010/0143301.
[53] Remington: The Science and Practice of Pharmacy (Gennaro, 2000; 20th edition, ISBN: 0683306472)
[54] WO2011/049677

1. (canceled)

2. An immunogenic composition comprising (i) a TLR7 agonist (ii) an insoluble metal salt and (iii) at least one S. aureus antigen selected from the group consisting of: a S. aureus EsxA antigen, a S. aureus EsxB antigen, a non-toxic S. aureus hemolysin mutant, a Sta006 antigen and a Sta011 antigen.

3. (canceled)

4. (canceled)

5. (canceled)

6. An immunogenic composition of claim 2 further comprising (iv) a buffer.

7. An immunogenic composition of claim 2 wherein the composition has a pH between 6 and 8.

8. (canceled)

9. The composition of claim 2, wherein the TLR7 agonist includes at least one adsorptive moiety which allows it to adsorb to insoluble metal salts.

10. The composition of claim 9, wherein the adsorptive moieties is a phosphate or a phosphonate.

11. The composition of claim 2, wherein the TLR7 agonist has formula (C), (D), (E), (F), (H), (I), (II) or (K), where formula (C), (D), (E) and (H) are: 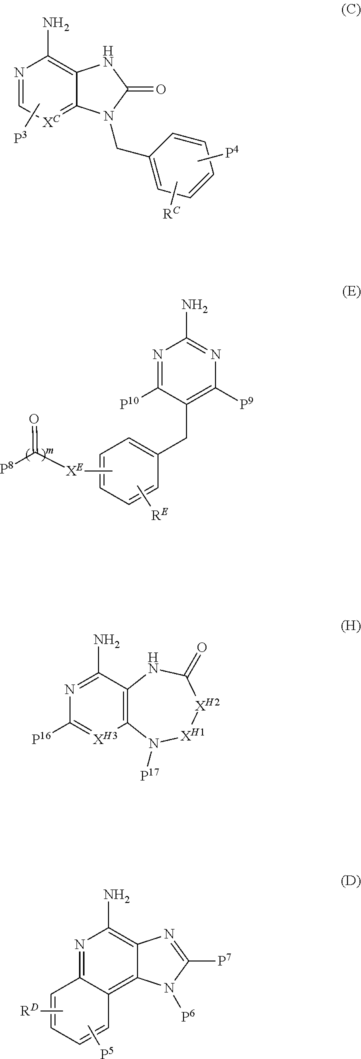
wherein:
(a) P³ is selected from H, C₁-C₆alkyl, CF₃, and —((CH₂)ₚO)_{q}(CH₂)ₚOₛ— and —Y-L-X—P(O)(OR^{X})(OR^{Y}); and P⁴ is selected from H, C₁-C₆alkyl, —C₁-C₆alkylaryl and —Y-L-X—P(O)(OR^{X})(OR^{Y}); with the proviso that at least one of P³ and P⁴ is —Y-L-X—P(O)(OR^{X})(OR^{Y}),
(b) P⁵ is selected from H, C₁-C₆alkyl, and —Y-L-X—P(O)(OR^{X})(OR^{Y}); P⁶ is selected from H, C₁-C₆alkyl each optionally substituted with 1 to 3 substituents selected from C₁-C₄alkyl and OH, and —Y-L-X—P(O)(OR^{X})(OR^{Y}); and P⁷ is selected from H, C₁-C₆alkyl, —((CH)ₚ(O)_{q}(CH₂)ₚOₛ—, —NHC₁-C₆alkyl and —Y-L-X—P(O)(OR^{X})(OR^{Y}); with the proviso that at least one of P⁵, P⁶ and P⁷ is —Y-L-X—P(O)(OR^{X})(OR^{Y});
(c) P⁸ is selected from H, C₁-C₆alkyl, C₁-C₆alkoxy, —NHC₁-C₆alkyl each optionally substituted with OH, and —Y-L-X—P(O)(OR^{X})(OR^{Y}); and P⁹ and P¹⁰ are each independently selected from H, C₁-C₆alkyl, C₁-C₆alkoxy, —NHC₁-C₆alkyl each optionally substituted with OH and C₁-C₆alkyl, and —Y-L-X—P(O)(OR^{X})(OR^{Y}); with the proviso that at least one of P⁸, P⁹ or P¹⁰ is —Y-L-X—P(O)(OR^{X})(OR^{Y});
(d) P¹⁶ and each P¹⁸ are each independently selected from H, C₁-C₆alkyl, and —Y-L-X—P(O)(OR^{X})(OR^{Y}); P¹⁷ is selected from H, C₁-C₆alkyl, aryl, heteroaryl, C₁-C₆alkylaryl, C₁-C₆alkyl heteroaryl, C₁-C₆alkylaryl-Y-L-X—P(O)(OR^{X})(OR^{Y}) and —Y-L-X—P(O)(OR^{X})(OR^{Y}), each optionally substituted with 1 to 2 substituents selected from C₁-C₆alkyl or heterocyclyl with the proviso that at least one of P¹⁶, P¹⁷ or a P¹⁸ contains a —Y-L-X—P(O)(OR^{X})(OR^{Y)} moiety;
R^{X} and R^{Y} are independently selected from H and C₁-C₆alkyl:
R^{C}, R^{D} and R^{H} are each independently selected from H and C₁-C₆alkyl:
X^{C} is selected from CH and N;
R^{E} is selected from H, C₁-C₆alkyl, C₁-C₆alkoxy, C(O)C₁-C₆alkyl, halogen and —((CH₂)ₚO)_{q}(CH₂)ₚ—;
X^{E} is selected from a covalent bond, CR^{E2}R^{E3} and NR^{E4};
R^{E2}, R^{E3} and R^{E4} are independently selected from H and C₁-C₆alkyl:
X^{H1}—X^{H2} is selected from —CR^{H2}R^{H3}, CR^{H2}R^{H3}—CR^{H2}R^{H3}—, —C(O)CR^{H2}R^{H3}—, —C(O)CR^{H2}R^{H3}—, —CR^{H2}R^{H3}C(O)—, —NR^{H4}C(O)—, C(O)NR^{H4}—, CR^{H2}R^{H3}S(O)₂ and —CR^{H2}═CR^{H2}—;
R^{H2}, R^{H3} and R^{H4} are each independently selected from H, C₁-C₆alkyl and P¹⁸;
X^{H3} is selected from N and CN;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
m is selected from 0 or 1;
each p is independently selected from 1, 2, 3, 4, 5 and 6;
q is selected from 1, 2, 3 and 4; and
s is selected from 0 and 1;
where formula (F) is: 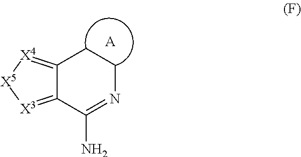
wherein:
X³ is N;
X⁴ is N or CR³
X⁵ is —CR⁴═CR⁵—;
R¹ and R² are H;
R³ is H;
R⁴ and R⁵ are each independently selected from H, halogen, —C(O)OR⁷, —C(O)R⁷, —C(O)N(R¹¹R¹²), —N(R¹¹R¹²), —N(R⁹)—NHN(R⁹)₂, —SR⁷, —(CH₂)ₙOR⁷, —(CH₂)ₙR⁷, -LR⁸, -LR¹⁰, —OLR⁸, —OLR¹⁰, C₁-C₆alkyl, C₁-C₆heteroalkyl, C₁-C₆haloalkyl, C₂-C₈alkene, C₇-C₈alkyne, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl groups of R⁴ and R⁵ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂, —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
or, R³ and R⁴, or R⁴ and R⁵, or R⁵ and R⁶, when present on adjacent ring atoms, can optionally be linked together to form a 5-6 membered ring, wherein the 5-6 membered ring is optionally substituted with R⁷;
each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₇-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂, —P(O)(OR¹⁰)₂, and —OP(O)(OR¹⁰)₂;
R⁷ is selected from H, C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl, wherein the C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl groups of R⁷ are each optionally substituted with 1 to 3 R¹³ groups, and each R¹³ is independently selected from halogen, —CN, -LR⁹, -LOR⁹, —OLR⁹, -LR¹⁰-LOR¹⁰, —OLR¹⁰, -LR⁸, -LOR⁸, —OLR⁸, -LSR⁸, -LSR¹⁰, -LC(O)R⁸, —OLC(O)R⁸, -LC(O)OR⁸, -LC(O)R¹⁰, -LOC(O)OR⁸, -LC(O)NR⁹R¹¹, -LC(O)NR⁹R⁸, -LN(R⁹)₂, -LNR⁹R⁸, -LNR⁹R¹⁰, -LC(O)N(R⁹)₂, -LS(O)₂R⁸, -LS(O)R⁸, -LC(O)NR⁸OH, -LNR⁹C(O)R⁸, -LNR⁹C(O)OR⁸, -LS(O)₂N(R⁹)₂, —OLS(O)₂N(R⁹)₂, -LNR⁹S(O)₂R⁸, -LC(O)NR⁹LN(R⁹)₂, -LP(O)(OR⁸)₂, -LOP(O)(OR⁸)₂, -LP(O)(OR¹⁰)₂ and —OLP(O)(OR¹⁰)₂;
each R⁸ is independently selected from H, —CH(R¹⁰)₂, C₁-C₈alkyl, C₂-C₈alkene, C₂-C₈alkyne, C₁-C₆haloalkyl, C₁-C₆alkoxy, C₁-C₆heteroalkyl, C₁-C₈cycloalkyl, C₂-C₈heterocycloalkyl, C₁-C₆hydroxyalkyl and C₁-C₆haloalkoxy, wherein the C₁-C₈alkyl, C₂-C₈alkene, C₇-C₈alkyne, C₁-C₆heteroalkyl, C₁-C₆haloalkyl, C₁-C₆alkoxy, C₃-C₈cycloalkyl, C₂-C₆heterocycloalkyl, C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹, —C(O)N(R⁹)₂, —C(O)OR¹¹, —NR⁹C(O)R¹¹, —NR⁹R¹⁰, —NR¹¹R¹², —N(R⁹)₂, —OR⁹, —OR¹⁰, —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂, and —OP(O)(OR¹¹)₂;
each R⁹ is independently selected from H, —C(O)R⁸, —C(O)OR⁸, —C(O)R¹⁰, —C(O)OR¹⁰, —S(O)₂R¹⁰, —C₁-C₆ alkyl, C₁-C₆ heteroalkyl and C₃-C₆cycloalkyl, or each R⁹ is independently a C₁-C₆alkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆cycloalkyl, or C₃-C₈heterocycloalkyl groups of R⁹ are each optionally substituted with 1 to 3 substituents independently selected from —CN, R¹¹, —OR¹¹, —SR¹¹, —C(O)R¹¹, OC(O)R¹¹, —C(O)OR¹¹, —NR¹¹R¹², —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂ and —OP(O)(OR¹¹)₂;
each R¹⁰ is independently selected from aryl, C₃-C₈cycloalkyl, C₃-C₈heterocycloalkyl and heteroaryl, wherein the aryl, C₃-C₈cycloalkyl, C₃-C₈heterocycloalkyl and heteroaryl groups are optionally substituted with 1 to 3 substituents selected from halogen, —R⁸, —OR⁸, -LR⁹, -LOR⁹, —N(R⁹)₂, NR⁹C(O)R⁸, —NR⁹CO₂R⁸.—CO₂R⁸, —C(O)R⁸ and —C(O)N(R⁹)₂;
R¹¹ and R¹² are independently selected from H, C₁-C₆alkyl, C₁-C₆heteroalkyl, C₁-C₆haloalkyl, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, aryl, heteroaryl, C₃-C₈cycloalkyl, and C₃-C₈heterocycloalkyl groups of R¹¹ and R¹² are each optionally substituted with 1 to 3 substituents independently selected from halogen, —CN, R⁸, —OR, C(O)R⁸, OC(O)R⁸, —C(O)OR⁸, —N(R⁹)₂, —NR⁸C(O)R⁸, —NR⁸C(O)OR⁸, —C(O)N(R⁹)₂, C₃-C₈heterocycloalkyl, —S(O)₂R⁸, —S(O)₂N(R⁹)₂, —NR⁹S(O)₂R⁸, C₁-C₆haloalkyl and C₁-C₆haloalkoxy;
or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
ring A is an aryl or a heteroaryl, wherein the aryl and heteroaryl groups of Ring A are optionally substituted with 1 to 3 R^{A} groups, wherein each R^{A} is independently selected from —R⁸, —R⁷, —OR⁷, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂, —NR⁹C(S)N(R⁹)₂, —NR⁹CO₂R⁸, —NR⁹NR⁹C(O)R⁸, —NR⁹NR⁹C(O)N(R⁹), —NR⁹NR⁹CO₂R⁸, —C(O)C(O)R⁸, —C(O)CH₂C(O)R⁸, —CO₂R⁸, —(CH₂)ₙCO₂R⁸, —C(O)R⁸, —C(S)R⁸, —C(O)N(R⁹), —C(S)N(R⁹)₂, —OC(O)N(R⁹)₂, —OC(O)R⁸, —C(O)N(OR⁸)R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸, —CH═CHCO₇R⁸, —C(═NH)—N(R⁹)₂, and —(CH₂)ₙNHC(O)R⁸ or two adjacent R^{A} substituents on Ring A form a 5-6 membered ring that contains up to two heteroatoms as ring members;
n is, independently at each occurrence, 0, 1, 2, 3, 4, 5, 6, 7 or 8;
each m is independently selected from 1, 2, 3, 4, 5 and 6, and
t is 1, 2, 3, 4, 5, 6, 7 or 8;
where formula (I) and (II) are: 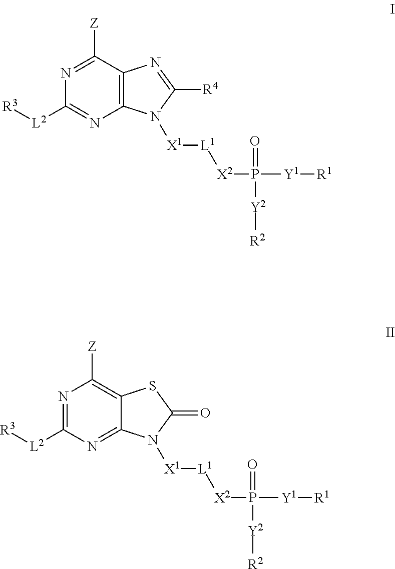
wherein:
Z is —NH₂ or —OH;
X¹ is alkylene, substituted alkylene, alkenylene, substituted alkenylene, alkynylene, substituted alkynylene, carbocyclylene, substituted carbocyclylene, heterocyclylene, or substituted heterocyclylene;
L¹ is a covalent bond, arylene, substituted arylene, heterocyclylene, substituted heterocyclylene, carbocyclylene, substituted carbocyclylene, —S—, —S(O)—, S(O)₂, —NR⁵—, or —O—
X² is a covalent bond, alkylene, or substituted alkylene;
L² is NR⁵—, —N(R⁵)C(O)—, —O—, —S—, —S(O)—, S(O)₂, or a covalent bond;
R³ is H, alkyl, substituted alkyl, heteroalkyl, substituted heteroalkyl, alkenyl, substituted alkenyl, aryl, substituted aryl, arylalkyl, substituted arylalkyl, heterocyclyl, substituted heterocyclyl, heterocyclylalkyl, or substituted heterocyclylalkyl;
Y¹ and Y² are each independently a covalent bond, —O— or —NR⁵—; or —Y¹—R¹ and —Y²—R² are each independently —O—N═C(R⁶R⁷);
R¹ and R² are each independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, substituted heterocyclylalkyl, -alkylene-C(O)—O—R⁵, -(substituted alkylene)-C(O)—O—R⁵, -alkylene-O—C(O)—R⁵, -(substituted alkylene)-O—C(O)—R⁵, -alkylene-O—C(O)—O—R⁵, or -(substituted alkylene)-O—C(O)—O—R⁵
R⁴ is H, halogen, —OH, —O-alkyl, —O-alkylene-O—C(O)—O—R⁵, —O—C(O)—O—R⁵, —SH, or —NH(R⁵);
each R⁵, R⁶, and R⁷ are independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, or substituted heterocyclylalkyl;
and where formula (K) is: 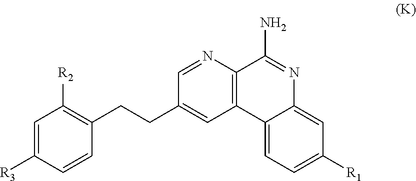
wherein:
R¹ is H, C₁-C₆alkyl, —C(R⁵)₂OH, -L¹R⁵, -L¹R⁶, -L²R⁶, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl:
R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₂P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

12. The composition of claim 2, wherein the TLR7 agonist is one of compounds 1 to 102 as defined in WO2012/031140, or a pharmaceutically acceptable salt thereof.

13. The composition of claim 2, wherein the TLR7 agonist is compound K2:

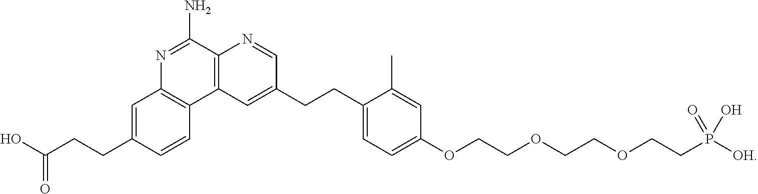 [SMILES: Cc1cc(OCCOCCOCCP(=O)(O)O)ccc1CCc1cnc2c(N)nc3cc(CCC(=O)O)ccc3c2c1]

14. The composition of claim 2, wherein the insoluble metal salt is an aluminium salt.

15. The composition of claim 14, wherein the aluminium salt is an aluminium hydroxide.

16. The composition of claim 14, having an Al⁺⁺⁺ concentration between 10-500 μg/ml.

17. The composition of claim 2, wherein >80% of the TLR7 agonist is adsorbed to the insoluble metal salt.

18. The composition of claim 6, wherein the buffer is a histidine buffer.

19. (canceled)

20. The composition of claim 2, including all four of: (i) a single polypeptide including both an EsxA antigen and an EsxB antigen; (ii) a Sta006 antigen; (iii) a Sta011 antigen; and (iv) a H35L mutant form of hemolysin.

21. The composition of claim 20, comprising:
an aluminium hydroxide adjuvant;
a TLR7 agonist of formula (K): 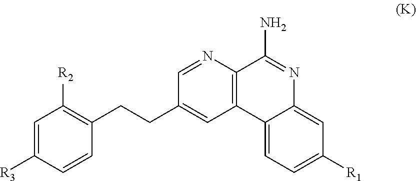
wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl:
R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₇P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4;
a first polypeptide comprising SEQ ID NO: 6, or a modified amino acid sequence which differs from SEQ ID NO: 6 by up to 5 single amino changes provided that the modified sequence can elicit antibodies which bind to a polypeptide consisting of SEQ ID NO: 6;
a second polypeptide comprising SEQ ID NO: 13, or a modified amino acid sequence which differs from SEQ ID NO: 13 by up to 5 single amino changes provided that the modified sequence can elicit antibodies which bind to a polypeptide consisting of SEQ ID NO: 13;
a third polypeptide comprising SEQ ID NO: 31, or a modified amino acid sequence which differs from SEQ ID NO: 31 by up to 5 single amino changes provided that the modified sequence can elicit antibodies which bind to a polypeptide consisting of SEQ ID NO: 31;
a fourth polypeptide comprising SEQ ID NO: 33, or a modified amino acid sequence which differs from SEQ ID NO: 33 by up to 5 single amino changes provided that the modified sequence can elicit antibodies which bind to a polypeptide consisting of SEQ ID NO: 33,
in which the TLR⁷ agonist and/or at least one of the polypeptides is/are adsorbed to the aluminium hydroxide adjuvant.

22. The composition of claim 21, comprising a first polypeptide having amino acid sequence SEQ ID NO: 7, a second polypeptide having amino acid sequence SEQ ID NO: 27, a third polypeptide having amino acid sequence SEQ ID NO: 32, and a fourth polypeptide having amino acid sequence SEQ ID NO: 8.

23. The composition of claim 21, comprising a first polypeptide having amino acid sequence SEQ ID NO: 44, a second polypeptide having amino acid sequence SEQ ID NO: 27, a third polypeptide having amino acid sequence SEQ ID NO: 45, and a fourth polypeptide having amino acid sequence SEQ ID NO: 46.

24. The composition of claim 21, wherein the TLR7 agonist of formula (K) is the following compound or a pharmaceutically acceptable salt thereof:

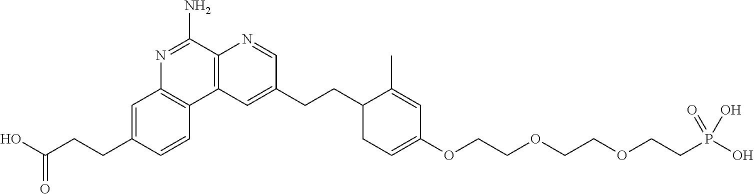 [SMILES: CC1=CC(OCCOCCOCCP(=O)(O)O)=CCC1CCc1cnc2c(N)nc3cc(CCC(=O)O)ccc3c2c1]

25. (canceled)

26. (canceled)

27. A method of raising an immune response in a subject, comprising the step of administering to the subject the composition of claim 2.

28. A process for preparing the immunogenic composition of claim 2, wherein the process comprises mixing a TLR7 agonist, an insoluble metal salt, and at least one S. aureus antigen selected from the group consisting of: a S. aureus EsxA antigen, a S. aureus EsxB antigen, a non-toxic S. aureus hemolysin mutant, a Sta006 antigen and a Sta011 antigen.

29. (canceled)

30. (canceled)

31. (canceled)

32. (canceled)

33. (canceled)

34. (canceled)

35. The composition of claim 20, wherein (i) the single polypeptide including both an EsxA antigen and an EsxB antigen comprises SEQ ID NO: 31; (ii) the Sta006 antigen comprises SEQ ID NO: 6; (iii) the Sta011 antigen comprises SEQ ID NO: 33; and (iv) the H35L mutant form of hemolysin comprises SEQ ID NO: 13.

36. The composition of claim 2, wherein the TLR7 agonist has formula (K′): 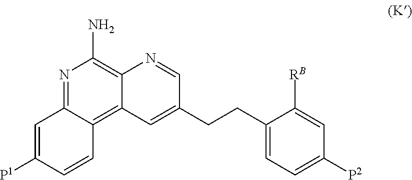
wherein:
P¹ is selected from H, C₁-C₆alkyl optionally substituted with COOH and —Y-L-X—P(O)(OR^{X})(OR^{Y});
P² is selected from H, C₁-C₆alkyl, C₁-C₆alkoxy and —Y-L-X—P(O)(OR^{X})(OR^{Y});
with the proviso that at least one of P¹ and P² is —Y-L-X—P(O)(OR^{X})(OR^{Y});
R^{B} is selected from H and C₁-C₆alkyl;
R^{X} and R^{Y} are independently selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and -((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6; and
q is selected from 1, 2, 3 and 4.

37. The composition of claim 2, wherein (iii) comprises a S. aureus EsxA antigen, a S. aureus EsxB antigen, a non-toxic S. aureus hemolysin mutant, a Sta006 antigen and a Sta011 antigen.

38. The composition of claim 22, wherein the TLR7 agonist of formula (K) is the following compound or a pharmaceutically acceptable salt thereof:

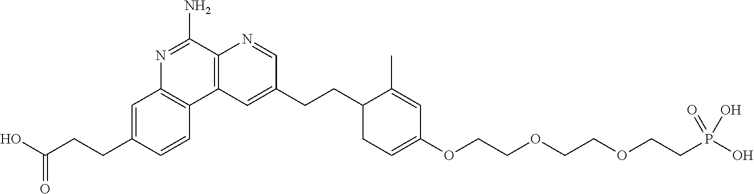 [SMILES: CC1=CC(OCCOCCOCCP(=O)(O)O)=CCC1CCc1cnc2c(N)nc3cc(CCC(=O)O)ccc3c2c1]

39. The composition of claim 23, wherein the TLR7 agonist of formula (K) is the following compound or a pharmaceutically acceptable salt thereof: 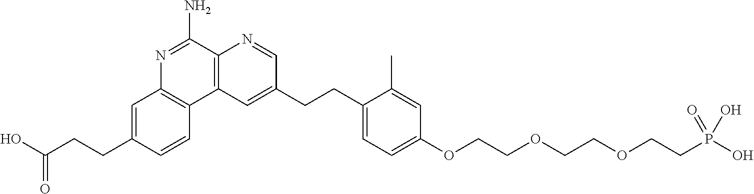 [SMILES: Cc1cc(OCCOCCOCCP(=O)(O)O)ccc1CCc1cnc2c(N)nc3cc(CCC(=O)O)ccc3c2c1]
